SEQUENCE LISTING (11 sequences)

SEQ ID NO: 1 - protein, 20 aa, Artificial Sequence
  P45 of apoB100
IEIGLEGKGFEPTLEALFGK

SEQ ID NO: 2 - protein, 14 aa, Artificial Sequence
  HCDR1
FSNAWMSWVRQAPG

SEQ ID NO: 3 - protein, 19 aa, Artificial Sequence
  HCDR2
SSISVGGHRTYYADSVKGR

SEQ ID NO: 4 - protein, 14 aa, Artificial Sequence
  HCDR3
ARIRVGPSGGAFDY

SEQ ID NO: 5 - protein, 14 aa, Artificial Sequence
  LCDR1
CSGSNTNIGKNYVS

SEQ ID NO: 6 - protein, 7 aa, Artificial Sequence
  LCDR2
ANSNRPS

SEQ ID NO: 7 - protein, 12 aa, Artificial Sequence
  LCDR3
CASWDASLNGWV

SEQ ID NO: 8 - protein, 120 aa, Artificial Sequence
  Variable heavy region
EVQLLESGGGLVQPGGSLRLSCAASGFTFSNAWMSWVRQAPGKGLEWVSSISVGGHRTYYADSVKGRSTISRDNSKNTLYLQMNSLRAEDTAVYYCARIRVGPSGGAFDYWGQGTLVTVS

SEQ ID NO: 9 - protein, 110 aa, Artificial Sequence
  Variable light region
QSVLTQPPSASGTPGQRVTISCSGSNTNIGKNYVSWYQQLPGTAPKLLIYANSNRPSGVPDRFSGSKSGTSASLAISGLRSEDEADYYCASWDASLNGWVFGGGTKLTVL

SEQ ID NO: 10 - protein, 451 aa, Artificial Sequence
  Heavy chain
EVQLLESGGGLVQPGGSLRLSCAASGFTFSNAWMSWVRQAPGKGLEWVSSISVGGHRTYYADSVKGRSTISRDNSKNTLYLQMNSLRAEDTAVYYCARIRVGPSGGAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 216 aa, Artificial Sequence
  Light chain
QSVLTQPPSASGTPGQRVTISCSGSNTNIGKNYVSWYQQLPGTAPKLLIYANSNRPSGVPDRFSGSKSGTSASLAISGLRSEDEADYYCASWDASLNGWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2021/0155682 A1
(43) Pub. Date: May 27, 2021

(54) COMPOSITIONS AND METHODS FOR REDUCTION OF LIPOPROTEIN A FORMATION AND TREATMENT OF AORTIC VALVE SCLEROSIS AND AORTIC STENOSIS

(71) Applicant: Abcentra, LLC, Los Angeles, CA (US)

(72) Inventor: Bertrand C. LIANG, San Diego, CA (US)

(21) Appl. No.: 17/257,055

(22) PCT Filed: Jul. 1, 2019
(86) PCT No.: PCT/US2019/040196

§ 371 Date: Dec. 30, 2020

Related U.S. Application Data
(60) Provisional application No. 62/697,353, filed on Jul. 12, 2018.
(60) Provisional application No. 62/693,218, filed on Jul. 2, 2018.

(51) Int. Cl. C07K 16/18; G01N 33/92

(52) U.S. Cl. CPC C07K 16/18; G01N 33/92; G01N 2500/02; C07K 2317/34; C07K 2317/76; C07K 2317/21

(57) ABSTRACT

Compositions and methods to reduce the formation of lipoprotein(a) are provided, thereby reducing the risk of aortic stenosis or aortic valve sclerosis. Antibodies or antigen-binding fragments thereof capable of binding apolipoprotein B₁₀₀ or apolipoprotein a are provided, which effectively prevents the binding and assembly of lipoprotein(a), thereby reducing the risk of aortic valve stenosis or sclerosis.

### CROSS-REFERENCE TO RELATED APPLICATIONS

This application includes a claim of priority under 35 U.S.C. § 119(e) to U.S. provisional patent application No. 62/693,218, filed Jul. 2, 2018, and to U.S. provisional patent application No. 62/697,353, filed Jul. 12, 2018, the entireties of which are hereby incorporated by reference.

### REFERENCE TO SEQUENCE LISTING

The Sequence Listing submitted Jul. 1, 2019, as a text file named “SequenceListing-070017-000029W000 ST25” created on Jun. 17, 2019 and having a size of 12,288 bytes, is hereby incorporated by reference.

### FIELD OF INVENTION

This invention relates to interventions and therapeutics for reducing the formation of lipoprotein(a).

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Coronary artery disease (CAD) is currently one of the most common causes of mortality and morbidity in developed and developing countries. Approximately 20% of the global population have elevated Lp(a), making it one of the most prevalent independent genetic risk marker for cardiovascular diseases. Elevated Lp(a) contributes to both the risk of a cardiovascular disease as well as to the accelerated exacerbation of aortic valve sclerosis (AVS). Aortic valve (AV) sclerosis (AVS) is a form of AV disease affecting an estimated 1 in 4 people above the age of 65 in the United States. An aging population and more widespread use of noninvasive imaging are increasing the incidence of AVS. AVS is typically defined as calcification of the aortic leaflets without impairment in leaflet excursion or a significant transvalvular pressure gradient. It is characterized by a gradual progression beginning with calcium deposition that may ultimately transform to aortic stenosis (AS) with obstruction of outflow from the left ventricle.

Lp(a) is a plasma lipoprotein, containing a cholesterol-rich, low-density lipoprotein (LDL)-like particle with one molecule of apolipoprotein B100 (ApoB100) and apolipoprotein (a) (apo(a)) attached via disulfide bonds, as depicted in FIG. 1. A distinctive difference between the structure of Lp(a) and LDL is the presence of the glycoprotein apo(a), which confers its characteristic properties on Lp(a) and is structurally similar to plasminogen, a precursor of plasmin, the fibrinolytic enzyme. This allows Lp(a) to bind to fibrin and to the membrane proteins of endothelial cells and monocytes. The primary site of synthesis of Lp(a) is the hepatocyte which also synthesizes ApoB100 Apo(a), on secretion, is then assembled with plasma LDL to form Lp(a) by the formation of a disulfide bond between ApoB100 in LDL and kringle IV in apo(a). The apolipoprotein (a) genotype alone accounts for 90% of the concentration in blood since it determines both the rate of synthesis as well as the size of the apo(a) moiety.

Since Lp(a) resembles both LDL and plasminogen, without being bound by theory it is hypothesized that it could act as a link between atherosclerosis and thrombosis. The accumulation of Lp(a) on the surface of fibrin and cell membranes as well as the inhibition of plasmin generation favors the deposition of fibrin and cholesterol at sites of vascular injury. After transfer into the arterial intima from the plasma, Lp(a) gets retained much more than LDL as it binds to the extracellular matrix through apo (a) as well as the apolipoprotein B component, thus contributing to the atherosclerotic plaque.

Lp(a) is an independent genetic risk marker for atherosclerosis and cardiovascular disease, as it has not been associated with other cardiac risk factors, such as cholesterol, LDL, HDL, triglycerides or C-reactive protein (CRP). There are few factors affecting Lp(a) levels in the blood. Its blood levels are genetically determined via variation in the apolipoprotein (a) gene (LPA). Thirty-four different isoforms of Lp(a) have been observed based on the size of apolipoprotein (a), and more than 90% of the inter-individual variation in plasma Lp(a) has been attributed to the apolipoprotein (a) gene, whilst 70% is related to the size of apolipoprotein (a) isoforms. Lp(a) levels are attained by the age of two. The high consistency of Lp(a) levels over time in a given individual indicates Lp(a) does not have significant correlation with either lifestyle modifications or any of the established cardiac risk factors.

Elevated Lp(a) presents a challenge for the management of the risk of cardiovascular diseases (CVDs). It has been found that at least 20% of the global population has Lp(a) levels in excess of 50 mg/dL, which is a significant risk factor for CVD. Indeed, it has recently been reported in a large database of over 500,000 patients referred for analysis of plasma lipids and other CVD biomarkers that 24% had levels>50 mg/Dl. In a tertiary care medical center database of 915 patients of particularly high CVD risk, 29.2% had levels>50 mg/dL. With current therapeutic approaches, it is very challenging to lower Lp(a), which poses a barrier to the clinical management of elevated Lp(a) and the understanding of a mechanistic etiology of Lp(a) in CVD.

Elevated Lp(a) and oxidized phospholipids-apoB levels are associated with faster aortic stenosis progression and need for aortic valve replacement. Several clinical studies have revealed Lp(a) and its associated oxidized phospholipids are causal, genetic risk factors for calcific aortic valve stenosis (CAVS).

Therefore, it is an objective of the present invention to provide compositions and methods for reducing the level of Lp(a) and/or treating and managing the risk of aortic stenosis, aortic valve sclerosis or both.

### SUMMARY OF THE INVENTION

The following embodiments and aspects thereof are described and illustrated in conjunction with compositions and methods which are meant to be exemplary and illustrative, not limiting in scope.

A method is provided for treating, reducing the severity of, slowing progression of or reducing the likelihood of aortic valve stenosis or aortic valve sclerosis in a subject in need thereof, through reducing or inhibiting the formation of lipoprotein(a) (Lp(a)). In one embodiment, the method includes administering to the subject an effective amount of a composition containing an antibody or antibody fragment capable of binding apolipoprotein B100 (ApoB100) or a fragment of ApoB100 (e.g., P45; SEQ ID NO:1), thereby reducing the association of ApoB100 (as a constituent of LDL) with apo(a) and as a result lowering the formation of Lp(a) by, for example, about 10%, 20%, 30%, 40% or 50%. In some embodiments, the method further includes selecting a subject with an elevated level of Lp(a) prior to administering the effective amount of the pharmaceutical composition. In some embodiments, the method further includes measuring the level of Lp(a) in the subject after the administration, and/or before the administration. In other embodiments, the subject is determined to have a reduced amount of lipoprotein(a) (Lp(a)) after the administration, determined to have an elevated amount of Lp(a) before the administration, or both. In some aspects, a reduced amount or level of Lp(a) after the administration of the antibody or antibody fragment is relative to the amount of the same subject before the administration, or relative to the amount from subjects not having aortic valve sclerosis or aortic stenosis or having been successfully treated from aortic valve sclerosis or aortic stenosis. In other aspects, an elevated amount or level of Lp(a) is relative to the amount or level of subject not having aortic valve sclerosis or aortic stenosis or having been successfully treated from aortic valve sclerosis or aortic stenosis.

Also provided are methods for reducing the level of lipoprotein(a) (Lp(a)) in a subject, which includes administering to the subject an antibody or antibody fragment that is capable of binding P45 fragment of ApoB100. Additional embodiments provide that the subject in the methods for reducing the level of Lp(a) is diagnosed with or shows symptoms of a cardiovascular disease, or aortic sclerosis and/or stenosis before the administration. Other embodiments provide the methods for reducing the level of Lp(a) further includes selecting a subject with an elevated level of Lp(a) before the administration. Additional aspects of the methods include measuring the level of Lp(a) in the subject after the administration, and/or before the administration; or the subject is determined to have a reduced amount of lipoprotein(a) (Lp(a)) after the administration.

Further embodiments provide that the antibody or antibody fragment in any of the disclosed methods comprises one, two or three heavy chain complementarity determining regions (HCDRs) selected from the group consisting of HCDR 1 (HCDR1), HCDR 2 (HCDR2) and HCDR 3 (HCDR3) sequences of SEQ ID Nos: 2, 3 and 4, respectively, and one, two or three light chain complementarity determining regions (LCDRs) selected from the group consisting of LCDR 1 (LCDR1), LCDR 2 (LCDR2) and LCDR 3 (LCDR3) sequences of SEQ ID Nos: 5, 6 and 7, respectively.

In one embodiment, a method for treating, reducing the severity of, slowing progression of or reducing the likelihood of aortic valve stenosis or aortic valve sclerosis in a subject in need thereof includes administering an effective amount of orticumab (also known as BI-204; MLDL 1278A; RG 7418, anti-oxLDL), thereby reducing or inhibiting the formation of Lp(a).

Also provided are methods for identifying a molecule or compound that reduces the binding between apolipoprotein (a) and low-density lipoprotein (LDL), and inhibits the formation of lipoprotein(a) (Lp(a)), which include contacting a molecule or compound of interest with a mixture of LDL and apolipoprotein (a); determining whether the contact between the molecule or compound of interest and the mixture results in a decrease in the binding between apolipoprotein (a) and LDL, a decrease in the amount of Lp(a), or both, compared to that in a mixture without the molecule or compound of interest, wherein a decrease in the binding between apolipoprotein (a) and LDL or a decrease in the amount of Lp(a) indicates that the molecule or compound of interest reduces the binding between apolipoprotein (a) and LDL, and inhibits the formation or reduces the amount of Lp(a).

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 is a schematic of the structure of lipoprotein (a) [Lp(a)], described in Manocha A, et al., Ind J Clin Biochem, 31:13 (2016).

FIG. 2A is a line graph depicting a titration curve of 50 nM fluorescein-labeled LDL with 17 kDa apolipoprotein (a) in the presence or absence of 1 μM orticumab (BI-204, “BI204”), or of 100 mM epsilon aminocaproic acid (EACA) (lysine analogue).

FIG. 2B depicts a line graph showing the titration of 17K apo(a) in the presence of 1000 nM orticumab, compared to 100 mM ε-aminocaproic acid (EACA) or no antibody (denoted “control”). Fluorescently-labeled LDL (Flu-LDL; 50 nM) was combined with 17K r-apo(a) (0, 10, 50, 100, 200, 500, or 1000 nM) in the presence of 1000 nM of orticumab or 100 mM EACA.

FIG. 2C depicts a line graph showing the titration of 17K apo(a) in the presence of 1000 nM orticumab, compared to up to 1000 nM anti-apo(a) polyclonal antibody or 100 mM EACA. The fluorescence observed in the absence of antibody, when quenching of the Flu-LDL fluorescence by apo(a) is lowest, was used as a correction factor in determining the fluorescence in the presence of antibodies and EACA. The lines in the graph depict non-linear regression of the data fit to a rectangular hyperbola.

FIG. 3 depicts titration of orticumab in the presence of fixed concentrations of Flu-LDL and apo(a). Fluorescently-labeled LDL (Flu-LDL; 50 nM) was combined with 17K r-apo(a) (500 nM) in the presence of orticumab (“BI”) at a concentration of 0, 0.064, 0.32, 1.6, 8, 40, 200, and 1000 nM; polyclonal anti-apoB-100 (ApoB) antibody was used as a positive control at the same concentrations; ε-aminocaproic acid (EACA) was also used as a positive control at 0, 0.0064, 0.032, 0.16, 0.8, 4, 20, and 100 mM. The absolute fluorescence value of Flu-LDL is represented by the upper dashed line, and the quenching effect of 17-K r-apo(a) is represented by the lower dotted line. The lines in the graph depict non-linear regression of the data fit to a rectangular hyperbola.

FIGS. 4A and 4B depict the titration of antibodies at lower concentrations with constant Flu-LDL and apo(a). Fluorescently-labeled LDL (Flu-LDL; 30 nM) was combined with 17K r-apo(a) (300 nM) in the presence of orticumab (“BI”) at a concentration of 0.4096, 1.024, 2.56, 6.4, 16, 40, and 100 nM (FIG. 4A). Polyclonal anti-apoB-100 (ApoB) antibody was used as a positive control at the same concentrations; and ε-aminocaproic acid (EACA) was also used as a positive control at 0.4096, 1.024, 2.56, 6.4, 16, 40, 100 mM (FIG. 4B). The absolute fluorescence value of Flu-LDL is represented by the upper dashed line, and the quenching effect of 17K r-apo(a) is represented by the lower dotted line.

FIG. 5 depicts antibody interference with Flu-LDL. Fluorescently-labeled LDL (Flu-LDL; 100 nM) was combined alone with orticumab (“BI”) at a concentration of 0.1, 1, 10, 100, and 1000 nM or 500 nM of either 17K or 17K Δ7,8. The absolute fluorescence value of Flu-LDL is shown with a dotted line (next to the label “Flu-LDL alone”), and the quenching effect of 17-K r-apo(a) and 17K Δ7,8 are shown in the lowest (next to the label “with 17K”) and the middle (next to the label “with 17K Δ7,8”) dashed lines, respectively.

FIG. 6 depicts western blots of Lp(a) covalent assembly over-time. 17K r-apo(a) (5 nM) was incubated with 100 nM LDL in serum free HEK293 cell-conditioned medium for 0, 2, 4, 6, and 8 hours at 37° C. The extent of covalent Lp(a) formation was assessed by Western blot analysis. The results are representative of three independent experiments.

FIG. 7 depicts Western blots of Lp(a) covalent assembly with antibody inhibition. 17K r-apo(a) (5 nM) was incubated with 100 nM LDL in serum free HEK293 cell-conditioned medium for 4 hours at 37° C. in the presence of the indicated concentrations of anti-apo(a), anti-apoB Abs, or orticumab. The extent of covalent Lp(a) formation was assessed by Western blot analysis.

FIG. 8 depicts Western blots of Lp(a) covalent assembly with antibody inhibition at other concentrations. 17K r-apo(a) (5 nM) was incubated with 100 nM LDL in serum free HEK293 cell-conditioned medium for 4 hours at 37° C. in the presence of the indicated concentrations of orticumab. Anti-apo(a) antibody was used as a control. The extent of covalent Lp(a) formation was assessed by Western blot analysis. The results are representative of three independent experiments.

FIG. 9 depicts Western blots of Lp(a) covalent assembly at 8 hours. 17K r-apo(a) (5 nM) was incubated with 100 nM LDL in serum free HEK293 cell-conditioned medium for 0 or 8 hours at 37° C. in the in the presence of indicated concentrations of orticumab. The extent of covalent Lp(a) formation was assessed by western blot analysis. The results are representative of three independent experiments. The band intensity of each Western blot was quantified in FIG. 10.

FIG. 10 depicts quantification of covalent Lp(a) assembly in the presence of varying concentrations of orticumab. Band intensity of Western blots (from 8-hour incubations) were quantified using ImageLab software (Bio-Rad) to calculate % r-Lp(a). Graphs and analysis were generated using Prism 7.0. The data shown are the means±SD of three independent experiments. *indicates p<0.05 versus no antibody (Two-Way ANOVA with Tukey post hoc test).

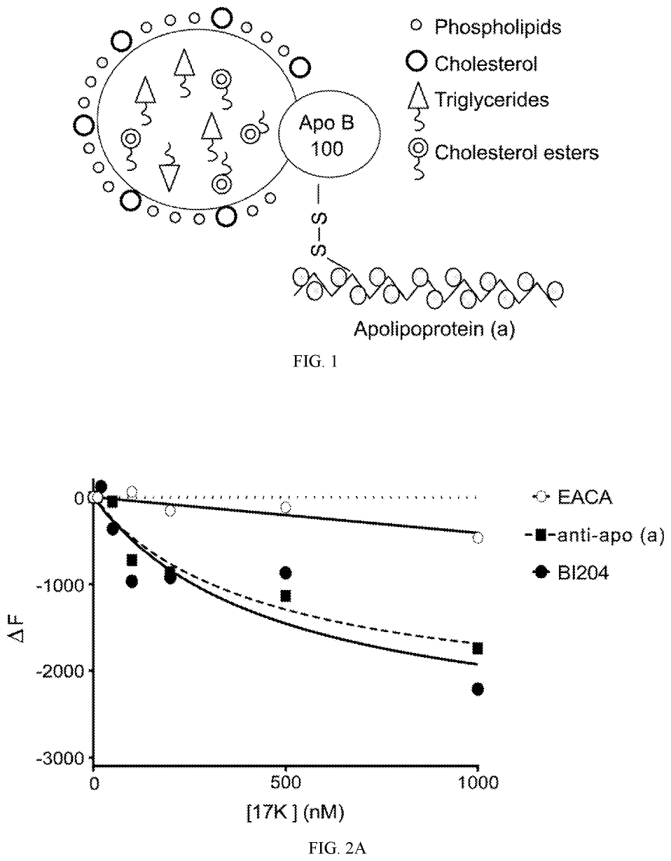

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, N.Y. 2006); March, Advanced Organic Chemistry Reactions, Mechanisms and Structure 7^{th} ed., J. Wiley & Sons (New York, N.Y. 2013); and Sambrook and Russel, Molecular Cloning: A Laboratory Manual 4^{th} ed., Cold Spring Harbor Laboratory Press (Cold Spring Harbor, N.Y. 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor N.Y., 2013); Kohler and Milstein, (1976) Eur. J. Immunol. 6: 511; Queen et al. U.S. Pat. No. 5,585,089; and Riechmann et al., Nature 332: 323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. September; 23(9):1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

The term “antibody” or “antibodies” as used herein are meant in a broad sense and includes immunoglobulin molecules including polyclonal antibodies, monoclonal antibodies including murine, human, human-adapted, humanized and chimeric monoclonal antibodies, antibody fragments, bispecific or multispecific antibodies, dimeric, tetrameric or multimeric antibodies, and single chain antibodies.

Immunoglobulins can be assigned to five major classes, namely IgA, IgD, IgE, IgG and IgM, depending on the heavy chain constant domain amino acid sequence. IgA and IgG are further sub-classified as the isotypes IgA₁, IgA₂, IgG₁, IgG₂, IgG₃ and IgG₄. Antibody light chains of any vertebrate species can be assigned to one of two clearly distinct types, namely kappa (κ) and lambda (λ), based on the amino acid sequences of their constant domains.

The term “antibody fragment” refers to a portion of an immunoglobulin molecule that retains the heavy chain and/or the light chain antigen binding site, such as heavy chain complementarity determining regions (HCDR) 1, 2 and 3, light chain complementarity determining regions (LCDR) 1, 2 and 3, a heavy chain variable region (V_{H}), or a light chain variable region (V_{L}). Antibody fragments include a Fab fragment, a monovalent fragment consisting of the V_{L}, V_{H}, C_{L} and C_{HI} domains; a F(ab)₂ fragment, a bivalent fragment comprising two Fab fragments linked by a disulfide bridge at the hinge region; a Fd fragment consisting of the V_{H} and C_{HI} domains; a Fv fragment consisting of the V_{L} and V_{H} domains of a single arm of an antibody; a domain antibody (dAb) fragment (Ward et al (1989) Nature 341:544-546), which consists of a V_{H} domain. V_{H} and V_{L} domains can be engineered and linked together via a synthetic linker to form various types of single chain antibody designs where the V_{H}/V_{L} domains pair intramolecularly, or intermolecularly in those cases when the V_{H} and V_{L} domains are expressed by separate single chain antibody constructs, to form a monovalent antigen binding site, such as single chain Fv (scFv) or diabody; described for example in PCT Intl. Publ. Nos. WO1998/44001, WO1988/01649, WO1994/13804, and WO1992/01047. These antibody fragments are obtained using well known techniques known to those of skill in the art, and the fragments are screened for utility in the same manner as are full length antibodies.

An antibody variable region consists of a “framework” region interrupted by three “antigen binding sites”. The antigen binding sites are defined using various terms such as Complementarity Determining Regions (CDRs), three in the V_{H} (HCDR1, HCDR2, HCDR3), and three in the V_{L} (LCDR1, LCDR2, LCDR3), are based on sequence variability (Wu and Kabat J Exp Med 132:211-50, 1970; Kabat et al Sequences of Proteins of Immunological Interest, 5th Ed. Public Health Service, National Institutes of Health, Bethesda, Md., 1991) or “Hypervariable regions”, “HVR”, or “HV”, three in the V_{H} (H1, H2, H3) and three in the V_{L} (L1, L2, L3), refer to the regions of an antibody variable domains which are hypervariable in structure as defined by Chothia and Lesk (Chothia and Lesk Mol Biol 196:901-17, 1987). Other terms include “IMGT-CDRs” (Lefranc et al., Dev Comparat Immunol 27:55-77, 2003) and “Specificity Determining Residue Usage” (SDRU) (Almagro, Mol Recognit 17:132-43, 2004). The International ImMunoGeneTics (IMGT) database provides a standardized numbering and definition of antigen-binding sites. The correspondence between CDRs, HVs and IMGT delineations is described in Lefranc et al., Dev Comparat Immuno! 27:55-77, 2003.

“Framework” or “framework sequences” are the remaining sequences of a variable region other than those defined to be antigen binding sites. Because the antigen binding sites can be defined by various terms as described above, the exact amino acid sequence of a framework depends on how the antigen-binding site was defined.

“Humanized antibody” refers to an antibody in which the antigen binding sites are derived from non-human species and the variable region frameworks are derived from human immunoglobulin sequences. Humanized antibodies may include substitutions in the framework regions so that the framework may not be an exact copy of expressed human immunoglobulin or germline gene sequences.

“Human-adapted” antibodies or “human framework adapted (HFA)” antibodies refer to humanized antibodies adapted according to methods described in U.S. Pat. Publ. No. US2009/0118127. Human-adapted antibodies are humanized by selecting the acceptor human frameworks based on the maximum CDR and FR similarities, length compatibilities and sequence similarities of CDR1 and CDR2 loops and a portion of light chain CDR3 loops.

“Human antibody” refers to an antibody having heavy and light chain variable regions in which both the framework and the antigen binding sites are derived from sequences of human origin. If the antibody contains a constant region, the constant region also is derived from sequences of human origin.

A human antibody comprises heavy or light chain variable regions that are “derived from” sequences of human origin wherein the variable regions of the antibody are obtained from a system that uses human germline immunoglobulin or rearranged immunoglobulin genes. Such systems include human immunoglobulin gene libraries displayed on phage, and transgenic non-human animals such as mice carrying human immunoglobulin loci as described herein. A “human antibody” may contain amino acid differences when compared to the human germline or rearranged immunoglobulin sequences due to for example naturally occurring somatic mutations or intentional introduction of substitutions in the framework or antigen binding sites. Typically, a human antibody is at least about 80%, 81%, 82%, 83%, 84%, 85%, 86%, 87%, 88%, 89%, 90%, 91%, 92%, 93%, 94%, 95%, 96%, 97%, 98%, 99% or 100% identical in amino acid sequence to an amino acid sequence encoded by a human germline or rearranged immunoglobulin gene. In some cases, “human antibody” may contain consensus framework sequences derived from human framework sequence analyses, for example as described in Knappik et al., J Mol Biol 296:57-86, 2000), or synthetic HCDR3 incorporated into human immunoglobulin gene libraries displayed on phage, for example as described in Shi et al., J Mol Biol 397:385-96, 2010 and Intl. Pat. Publ. No. WO2009/085462. Antibodies in which antigen binding sites are derived from a non-human species are not included in the definition of human antibody.

The term “recombinant antibody” as used herein, includes all antibodies that are prepared, expressed, created or isolated by recombinant means, such as antibodies isolated from an animal (e.g., a mouse) that is transgenic or transchromosomal for human immunoglobulin genes or a hybridoma prepared therefrom (described further below), antibodies isolated from a host cell transformed to express the antibody, antibodies isolated from a recombinant, combinatorial antibody library, and antibodies prepared, expressed, created or isolated by any other means that involve splicing of human immunoglobulin gene sequences to other DNA sequences, or antibodies that are generated in vitro using Fab arm exchange such as bispecific antibodies.

The term “monoclonal antibody” as used herein refers to a preparation of antibody molecules of single molecular composition. A monoclonal antibody composition displays a single binding specificity and affinity for a particular epitope, or in a case of a bispecific monoclonal antibody, a dual binding specificity to two distinct epitopes.

The term “epitope” as used herein means a portion of an antigen to which an antibody specifically binds. Epitopes usually consist of chemically active (such as polar, non-polar or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

“Variant” as used herein refers to a polypeptide or a polynucleotide that differs from a reference polypeptide or a reference polynucleotide by one or more modifications for example, substitutions, insertions or deletions.

“Administering” and/or “administer” as used herein refer to any route for delivering a pharmaceutical composition to a patient. Routes of delivery may include non-invasive peroral (through the mouth), topical (skin), transmucosal (nasal, buccal/sublingual, vaginal, ocular and rectal) and inhalation routes, as well as parenteral routes, and other methods known in the art. Parenteral refers to a route of delivery that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, sub arachnoid, subcapsular, subcutaneous, transmucosal, or transtracheal. Via the parenteral route, the compositions may be in the form of solutions or suspensions for infusion or for injection, or as lyophilized powders.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and/or prolonging a patient's life or life expectancy. In some embodiments, the disease condition is rheumatoid arthritis, or a combination of rheumatoid arthritis and accelerated atherosclerosis.

The term “effective amount” as used herein refers to the amount of a pharmaceutical composition comprising one or more antibodies or peptides as disclosed herein or a mutant, variant, analog or derivative thereof, to decrease at least one or more symptom of the disease or disorder, and relates to a sufficient amount of pharmacological composition to provide the desired effect. The phrase “therapeutically effective amount” as used herein means a sufficient amount of the composition to treat a disorder, at a reasonable benefit/risk ratio applicable to any medical treatment. In one embodiment, the pharmaceutical (therapeutic) composition comprises, consists of or consists essentially of an antibody against ApoB100. In various embodiments, the pharmaceutical compositions described herein further comprise a pharmaceutically acceptable carrier. In some embodiments, a therapeutic pharmaceutical composition is used, for example, to treat, inhibit, reduce the severity of and/or, reduce duration of a cardiovascular disease, such as atherosclerosis or thrombosis, and/or related symptoms in a subject in need thereof.

A therapeutically or prophylactically significant reduction in a symptom is, e.g. at least about 10%, at least about 20%, at least about 30%, at least about 40%, at least about 50%, at least about 60%, at least about 70%, at least about 80%, at least about 90%, at least about 100%, or more in a measured parameter as compared to a control or non-treated subject or the state of the subject prior to administering the compositions described herein. Measured or measurable parameters include clinically detectable markers of disease, for example, elevated or depressed levels of a biological marker, as well as parameters related to a clinically accepted scale of symptoms or markers for rheumatoid arthritis and/or accelerated atherosclerosis. It will be understood, however, that the total daily usage of the compositions and formulations as disclosed herein will be decided by the attending physician within the scope of sound medical judgment. The exact amount required will vary depending on factors such as the type of disease being treated, gender, age, and weight of the subject.

“Subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the mammal is a human subject.

The terms “treat,” “treatment,” “treating,” or “amelioration” when used in reference to a disease, disorder or medical condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease-state is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented.

The term “statistically significant” or “significantly” refers to statistical evidence that there is a difference. It is defined as the probability of making a decision to reject the null hypothesis when the null hypothesis is actually true. The decision is often made using the p-value.

“Selectively binds” or “specifically binds” refers to the ability of an antibody or antibody fragment thereof described herein to bind to a target, such as a molecule present on the cell-surface, with a K_{D} 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay.

A “cardiovascular disease,” as used herein, refers to a disorder of the heart and blood vessels, and includes disorders of the arteries, veins, arterioles, venules, and capillaries. Non-limiting examples of cardiovascular diseases include congestive heart failure, arrhythmia, pericarditis, acute myocardial infarction, infarcted myocardium, coronary artery disease, coronary heart disease, ischemic heart disease, cardiomyopathy, stroke, hypertensive heart disease, heart failure, pulmonary heart disease, ischemic syndrome, coronary microvascular disease, cardiac dysrhythmias, rheumatic heart disease, aortic aneurysms, atrial fibrillation, congenital heart disease, endocarditis, inflammatory heart disease, endocarditis, inflammatory cardiomegaly, myocarditis, valvular heart disease, cerebrovascular disease, and peripheral artery disease, or any combination thereof. In some embodiments, the cardiovascular disease to be treated by the disclosed methods includes aortic valve stenosis or aortic valve sclerosis.

“Aortic sclerosis” is the deposition of calcium and thickening of the aortic wall or aortic valve. “Aortic valve sclerosis” refers to the deposition of calcium and thickening of the aortic valve, typically in the absence of obstruction of ventricular outflow. Clinically, aortic valve sclerosis can be suspected in the presence of symptoms such as soft ejection systolic murmur at the aortic area, normal split of the second heart sound, and normal volume carotid pulse, but it can be best detected by echocardiography.

“Aortic stenosis” refers to increased blood flow velocity across a narrowed valve orifice, which is a common cause of left ventricular outflow tract obstruction. A common cause of aortic stenosis is calcific valvular disease, followed by congenital bicuspic aortic valve. Another common cause if rheumatic heart disease. Aortic stenosis is usually suspected on the basis of a systolic murmur on routine cardiac examination. The presence of the following findings can indicate the likelihood of severe aortic stenosis: long ejection systolic murmur with radiation to carotids; delayed carotid upstroke; single or paradoxical splitting of second heart sound. Transthoracic echocardiography (TTE) is commonly used in diagnosing aortic stenosis.

The term “in combination with” as used herein means that two or more therapeutics can be administered to a subject together in a mixture, concurrently as single agents or sequentially as single agents in any order.

Methods and Systems

Various embodiments provide methods for treating, reducing the severity of, slowing progression of or reducing the likelihood of aortic sclerosis (e.g., aortic valve sclerosis) or aortic stenosis in a subject by administering to the subject a pharmaceutical composition that includes an antibody or antibody fragment that binds to at least one fragment of apolipoprotein B100 (apoB100) and lowering the level of Lp(a) in the subject.

Various embodiments provide methods for reducing the level of lipoprotein(a) (Lp(a)) in a subject, optionally the subject being diagnosed with or showing symptoms of a cardiovascular disease, which includes administering to the subject a pharmaceutical composition that contains an antibody or antibody fragment that binds to at least one fragment of apolipoprotein B100 (apoB100). Further aspects of these embodiments provide that the subject is diagnosed with or showing symptoms of aortic sclerosis and/or aortic stenosis. Yet further aspects of the methods include identifying or selecting a subject with an elevated level of Lp(a) and/or exhibiting symptoms of aortic valve sclerosis and/or aortic stenosis, then administering to the subject the effective amount of the antibody or antibody fragment. Additional aspects include quantifying the level of Lp(a) and/or symptoms of aortic valve sclerosis or aortic stenosis after the administration, and if the level of Lp(a) or the symptoms persist, continue administering the antibody or antibody fragment.

Various embodiments provide the antibody or antibody fragment in the methods disclosed herein binds to a native and/or an oxidized epitope P45 of apoB100. Various embodiments provide the antibody or antibody fragment in the methods disclosed herein only binds to a native and/or an oxidized epitope P45 of apoB100. P45 of apoB100 has a polypeptide sequence of IEIGLEGKGFEPTLEALFGK (SEQ ID No.: 1). An oxidized epitope or oxidized lipoprotein includes but is not limited to a modification on the epitope or lipoprotein to carry malone-di-aldehyde (MDA) groups on lysines and histidines, a modification that is induced by oxidation by copper (e.g., CuOxLDL), a modification to carry hydroxynonenal, or a modification to carry a hapten of an aldehyde. Another embodiment provides the antibody or antibody fragment in the method disclosed herein further binds one or more fragments of apoB100.

ApoB100 contains peptide fragments that can be identified as P1-P302, which have overlapping amino acids between adjacent peptides, as described in U.S. patent application publication no. US/2017/0340702 and U.S. Pat. Nos. 7,468,183 and 7,704,499, which are incorporated by reference herein in their entireties.

Various embodiments provide that the method of treating, reducing the severity or likelihood of aortic sclerosis and/or aortic stenosis in a subject includes but is not limited to administering orticumab or a variant of orticumab that has identical heavy chain and/or light chain to those of orticumab, or identical complementarity determining regions to those of orticumab.

Various embodiments provide that the method of lowering the level of Lp(a) in a subject includes but is not limited to administering orticumab or a variant of orticumab that has identical heavy chain and/or light chain to those of orticumab, or identical complementarity determining regions to those of orticumab. Further aspects of the embodiments include that the subject is diagnosed with or shows symptoms of aortic sclerosis or aortic stenosis before the administration, and the symptoms improve after the administration. further aspects of the methods include identifying or selecting a subject with an elevated level of Lp(a) and/or exhibiting symptoms of aortic valve sclerosis and/or aortic stenosis, then administering to the subject the effective amount of the antibody or antibody fragment.

Orticumab is a human monoclonal antibody that contains heavy chain complementarity determining regions (HCDR) 1 (HCDR1), 2 (HCDR2) and 3 (HCDR3) as set forth in SEQ ID Nos: 2, 3 and 4, respectively; and light chain complementarity determining regions (LCDR) 1 (LCDR1), 2 (LCDR2) and 3 (LCDR3) as set forth in SEQ ID Nos: 5, 6 and 7, respectively. Orticumab contains a variable heavy region (V_{H}) amino acid sequence of SEQ ID No: 8, a variable light region (V_{L}) amino acid sequence of SEQ ID No: 9. Orticumab contains a heavy chain amino acid sequence of SEQ ID No: 10, a light chain amino acid sequence of SEQ ID No: 11. The amino acid sequence of orticumab is also described in WO2009/083225 and U.S. Patent Application Publication No. US20110014203, which are incorporated by reference herein in their entireties.

HCDR1, i.e., SEQ ID No.: 2, is:
FSNAWMSWVRQAPG.
HCDR2, i.e., SEQ ID No.: 3, is:
SSISVGGHRTYYADSVKGR.
HCDR3, i.e., SEQ ID No.: 4, is:
ARIRVGPSGGAFDY.
LCDR1, i.e., SEQ ID No.: 5, is:
CSGSNTNIGKNYVS.
LCDR2, i.e., SEQ ID No.: 6, is:
ANSNRPS.
LCDR3, i.e., SEQ ID No.: 7, is:
CASWDASLNGWV.

Variable heavy region (V_{H}), i.e., SEQ ID No.:8, is as shown:

EVQLLESGGG LVQPGGSLRL SCAASGFTFS NAWMSWVRQA
PGKGLEWVSS ISVGGHRTYY ADSVKGRSTI SRDNSKNTLY
LQMNSLRAED TAVYYCARIR VGPSGGAFDY WGQGTLVTVS.

Variable light region (V_{L}), i.e., SEQ ID No.: 9, is as shown:

QSVLTQPPSA SGTPGQRVTI SCSGSNTNIG KNYVSWYQQL
PGTAPKLLIY ANSNRPSGVP DRFSGSKSGT SASLAISGLR
SEDEADYYCA SWDASLNGWV FGGGTKLTVL.

Heavy chain, i.e., SEQ ID No.:10, is as shown:

EVQLLESGGG LVQPGGSLRL SCAASGFTFS NAWMSWVRQA
PGKGLEWVSS ISVGGHRTYY ADSVKGRSTI SRDNSKNTLY
LQMNSLRAED TAVYYCARIR VGPSGGAFDY WGQGTLVTVS
SASTKGPSVF PLAPSSKSTS GGTAALGCLV KDYFPEPVTV
SWNSGALTSG VHTFPAVLQS SGLYSLSSVV TVPSSSLGTQ
TYICNVNHKP SNTKVDKKVE PKSCDKTHTC PPCPAPELLG
GPSVFLFPPK PKDTLMISRT PEVTCVVVDV SHEDPEVKFN
WYVDGVEVHN AKTKPREEQY NSTYRVVSVL TVLHQDWLNG
KEYKCKVSNK ALPAPIEKTI SKAKGQPREP QVYTLPPSRD
ELTKNQVSLT CLVKGFYPSD IAVEWESNGQ PENNYKTTPP
VLDSDGSFFL YSKLTVDKSR WQQGNVFSCS VMHEALHNHY
TQKSLSLSPG K.

Light chain, i.e., SEQ ID No.:11, is as shown:

QSVLTQPPSA SGTPGQRVTI SCSGSNTNIG KNYVSWYQQL
PGTAPKLLIY ANSNRPSGVP DRFSGSKSGT SASLAISGLR
SEDEADYYCA SWDASLNGWV FGGGTKLTVL GQPKAAPSVT
LFPPSSEELQ ANKATLVCLI SDFYPGAVTV AWKADSSPVK
AGVETTTPSK QSNNKYAASS YLSLTPEQWK SHRSYSCQVT
HEGSTVEKTV APTECS.

Methods are provided of treating or reducing the severity or likelihood of aortic valve sclerosis or aortic stenosis, and/or lowering the level of Lp(a), in a subject including administering to the subject an effective amount of an antibody or antibody fragment that binds a fragment set forth in SEQ ID No.: 1 of apoB100, and the antibody contains one or more of HCDR1, HCDR2, HCDR3, LCDR1, LCDR2 and LCDR3 as set forth in SEQ ID Nos.: 2-7, respectively; optionally including selecting a subject with an elevated level of Lp(a) or showing symptoms of aortic valve sclerosis or aortic stenosis before the administration.

The antibody containing “one or more of HCDR1, HCDR2, HCDR3, LCDR1, LCDR2 and LCDR3” encompasses embodiments that the antibody contains one, any two, any three, any four, any five or all six of the CDRs (i.e., HCDR1, HCDR2, HCDR3, LCDR1, LCDR2 and LCDR3). One aspect of the invention provides an antibody comprising at least one complementarity determining region (CDR) that has the amino acid sequence of the corresponding CDR of antibody orticumab; that more preferably, the antibody has two or three or four or five CDRs that have the sequence of the corresponding CDRs of antibody orticumab; that if the antibody has three or four CDRs that have the sequence of the corresponding CDRs of antibody orticumab, it is preferred if the antibody has all three heavy chain or all three light chain CDRs that have the sequence of the corresponding CDRs of antibody orticumab; that thus this aspect of the invention includes an antibody comprising three light chain CDRs that have the sequence of the corresponding three light chain CDRs of antibody orticumab, or three heavy chain CDRs that have the sequence of the corresponding three heavy chain CDRs of antibody orticumab; that yet more preferably, the antibody comprises three light chain CDRs and three heavy chain CDRs that have the sequence of the corresponding CDRs of antibody orticumab; that if the antibody does not comprise all six CDRs that have the sequence of the corresponding CDRs of antibody orticumab, it is preferred if some or all of the 1, 2, 3, 4 or 5 “non-identical” CDRs comprise a variant of the sequence of the corresponding CDRs of antibody orticumab, (by “a variant” we includes the meaning that the variant has at least 50% sequence identity with the sequence of the corresponding CDR, more preferably at least 70%, yet more preferably at least 80% or at least 90% or at least 95%; most preferably, the variant has 96% or 97% or 98% or 99% sequence identity with the sequence of the corresponding CDR of antibody orticumab; typically the “variant” CDR sequence has 5 or 4 or 3 or 2 or only 1 amino acid residue difference from the sequence of the corresponding CDR of antibody orticumab); and that this aspect of the invention includes antibody orticumab. For example, one aspect of the embodiment provides that the administered antibody contains HCDR1 as set forth in SEQ ID No.: 2. Another aspect provides that the administered antibody contains HCDR2 as set forth in SEQ ID No.: 3. Another aspect provides that the administered antibody contains HCDR3 as set forth in SEQ ID No.: 4. Yet another aspect provides that the administered antibody contains LCDR1 as set forth in SEQ ID No.: 5. Another aspect provides that the administered antibody contains LCDR2 as set forth in SEQ ID No.: 6. Another aspect provides that the administered antibody contains LCDR3 as set forth in SEQ ID No.:7. Yet another aspect provides that the administered antibody contains HCDR1 as set forth in SEQ ID No.:2 and HCDR2 as set forth in SEQ ID No.: 3. Another aspect provides that the administered antibody contains HCDR1 as set forth in SEQ ID No.:2 and HCDR3 as set forth in SEQ ID No.: 4. Another aspect provides that the administered antibody contains HCDR1 as set forth in SEQ ID No.:2 and LCDR1 as set forth in SEQ ID No.: 5. Another aspect provides that the administered antibody contains HCDR1 as set forth in SEQ ID No.:2 and LCDR2 as set forth in SEQ ID No.: 6. Another aspect provides that the administered antibody contains HCDR1 as set forth in SEQ ID No.:2 and LCDR3 as set forth in SEQ ID No.: 7. Another aspect provides that the administered antibody contains HCDR2 as set forth in SEQ ID No.:3 and HCDR3 as set forth in SEQ ID No.: 4. Another aspect provides that the administered antibody contains HCDR2 as set forth in SEQ ID No.:3 and LCDR1 as set forth in SEQ ID No.: 5. Another aspect provides that the administered antibody contains HCDR2 as set forth in SEQ ID No.:3 and LCDR2 as set forth in SEQ ID No.: 6. Another aspect provides that the administered antibody contains HCDR2 as set forth in SEQ ID No.:3 and LCDR3 as set forth in SEQ ID No.: 7. Another aspect provides that the administered antibody contains HCDR3 as set forth in SEQ ID No.:4 and LCDR1 as set forth in SEQ ID No.: 5. Another aspect provides that the administered antibody contains HCDR3 as set forth in SEQ ID No.:4 and LCDR2 as set forth in SEQ ID No.: 6. Another aspect provides that the administered antibody contains HCDR3 as set forth in SEQ ID No.:4 and LCDR3 as set forth in SEQ ID No.: 7. Another aspect provides that the administered antibody contains LCDR1 as set forth in SEQ ID No.:5 and LCDR2 as set forth in SEQ ID No.: 6. Another aspect provides that the administered antibody contains LCDR1 as set forth in SEQ ID No.:5 and LCDR3 as set forth in SEQ ID No.: 7. Another aspect provides that the administered antibody contains LCDR2 as set forth in SEQ ID No.:6 and LCDR3 as set forth in SEQ ID No.: 7. Another aspect provides that the administered antibody contains HCDR1, HCDR2 and HCDR3 as set forth in SEQ ID Nos.: 2-4, respectively. Another aspect provides that the administered antibody contains HCDR1, HCDR2 and LCDR1 as set forth in SEQ ID Nos.: 2, 3 and 5, respectively. Another aspect provides that the administered antibody contains HCDR1, HCDR2 and LCDR2 as set forth in SEQ ID Nos.: 2, 3 and 6, respectively. Another aspect provides that the administered antibody contains HCDR1, HCDR2 and LCDR3 as set forth in SEQ ID Nos.: 2, 3 and 7, respectively. Another aspect provides that the administered antibody contains HCDR1, HCDR3 and LCDR1 as set forth in SEQ ID Nos.: 2, 4 and 5, respectively. Another aspect provides that the administered antibody contains HCDR1, HCDR3 and LCDR2 as set forth in SEQ ID Nos.: 2, 4 and 6, respectively. Another aspect provides that the administered antibody contains HCDR1, HCDR3 and LCDR3 as set forth in SEQ ID Nos.: 2, 4 and 7, respectively. Another aspect provides that the administered antibody contains HCDR1, LCDR1 and LCDR2 as set forth in SEQ ID Nos.: 2, 5 and 6, respectively. Another aspect provides that the administered antibody contains HCDR1, LCDR1 and LCDR3 as set forth in SEQ ID Nos.: 2, 5 and 7, respectively. Another aspect provides that the administered antibody contains HCDR1, LCDR2 and LCDR3 as set forth in SEQ ID Nos.: 2, 6 and 7, respectively. Another aspect provides that the administered antibody contains HCDR2, HCDR3 and LCDR1 as set forth in SEQ ID Nos.: 3, 4 and 5, respectively. Another aspect provides that the administered antibody contains HCDR2, HCDR3 and LCDR2 as set forth in SEQ ID Nos.: 3, 4 and 6, respectively. Another aspect provides that the administered antibody contains HCDR2, HCDR3 and LCDR3 as set forth in SEQ ID Nos.: 3, 4 and 7, respectively. Another aspect provides that the administered antibody contains HCDR2, LCDR1 and LCDR2 as set forth in SEQ ID Nos.: 3, 5 and 6, respectively. Another aspect provides that the administered antibody contains HCDR2, LCDR1 and LCDR3 as set forth in SEQ ID Nos.: 3, 5 and 7, respectively. Another aspect provides that the administered antibody contains HCDR2, LCDR2 and LCDR3 as set forth in SEQ ID Nos.: 3, 6 and 7, respectively. Another aspect provides that the administered antibody contains HCDR3, LCDR1 and LCDR2 as set forth in SEQ ID Nos.:4, 5 and 6, respectively. Another aspect provides that the administered antibody contains HCDR3, LCDR1 and LCDR3 as set forth in SEQ ID Nos.:4, 5 and 7, respectively. Another aspect provides that the administered antibody contains HCDR3, LCDR2 and LCDR3 as set forth in SEQ ID Nos.:4, 6 and 7, respectively. Another aspect provides that the administered antibody contains LCDR1, LCDR2 and LCDR3 as set forth in SEQ ID Nos.:5-7, respectively. Yet another aspect provides that the administered antibody contains HCDR1, HCDR2, HCDR3 and LCDR1 as set forth in SEQ ID Nos.:2-5, respectively. Another aspect provides that the administered antibody contains HCDR1, HCDR2, HCDR3 and LCDR2 as set forth in SEQ ID Nos.:2-4 and 6, respectively. Another aspect provides that the administered antibody contains HCDR1, HCDR2, HCDR3 and LCDR3 as set forth in SEQ ID Nos.:2-4 and 7, respectively. Another aspect provides that the administered antibody contains HCDR1, HCDR2, LCDR1 and LCDR2 as set forth in SEQ ID Nos.:2, 3, 5 and 6, respectively. Another aspect provides that the administered antibody contains HCDR1, HCDR2, LCDR1 and LCDR3 as set forth in SEQ ID Nos.:2, 3, 5 and 7, respectively. Another aspect provides that the administered antibody contains HCDR1, HCDR2, LCDR2 and LCDR3 as set forth in SEQ ID Nos.:2, 3, 6 and 7, respectively. Another aspect provides that the administered antibody contains HCDR1, HCDR3, LCDR1 and LCDR2 as set forth in SEQ ID Nos.:2, 4, 5 and 6, respectively. Another aspect provides that the administered antibody contains HCDR1, HCDR3, LCDR1 and LCDR3 as set forth in SEQ ID Nos.:2, 4, 5 and 7, respectively. Another aspect provides that the administered antibody contains HCDR1, HCDR3, LCDR2 and LCDR3 as set forth in SEQ ID Nos.:2, 4, 6 and 7, respectively. Another aspect provides that the administered antibody contains HCDR1, LCDR1, LCDR2 and LCDR3 as set forth in SEQ ID Nos.:2, 5, 6 and 7, respectively. Another aspect provides that the administered antibody contains HCDR2, HCDR3, LCDR1 and LCDR2 as set forth in SEQ ID Nos.:3-6, respectively. Another aspect provides that the administered antibody contains HCDR2, HCDR3, LCDR1 and LCDR3 as set forth in SEQ ID Nos.:3-5 and 7, respectively. Another aspect provides that the administered antibody contains HCDR2, HCDR3, LCDR2 and LCDR3 as set forth in SEQ ID Nos.:3, 4, 6 and 7, respectively. Another aspect provides that the administered antibody contains HCDR2, LCDR1, LCDR2 and LCDR3 as set forth in SEQ ID Nos.:3, 5, 6 and 7, respectively. Another aspect provides that the administered antibody contains HCDR3, LCDR1, LCDR2 and LCDR3 as set forth in SEQ ID Nos.:4, 5, 6 and 7, respectively. Yet another aspect provides that the administered antibody contains HCDR1, HCDR2, HCDR3, LCDR1 and LCDR2 as set forth in SEQ ID Nos.: 2-6 respectively. Another aspect provides that the administered antibody contains HCDR1, HCDR2, HCDR3, LCDR1 and LCDR3 as set forth in SEQ ID Nos.: 2-5 and 7 respectively. Another aspect provides that the administered antibody contains HCDR1, HCDR2, HCDR3, LCDR2 and LCDR3 as set forth in SEQ ID Nos.: 2, 3, 4, 6 and 7 respectively. Another aspect provides that the administered antibody contains HCDR1, HCDR2, LCDR1, LCDR2 and LCDR3 as set forth in SEQ ID Nos.: 2, 3, 5-7, respectively. Another aspect provides that the administered antibody contains HCDR1, HCDR3, LCDR1, LCDR2 and LCDR3 as set forth in SEQ ID Nos.: 2, 4-7, respectively. Another aspect provides that the administered antibody contains HCDR2, HCDR3, LCDR1, LCDR2 and LCDR3 as set forth in SEQ ID Nos.: 3-7, respectively. Yet another aspect provides that the administered antibody contains HCDR1, HCDR2, HCDR3, LCDR1, LCDR2 and LCDR3 as set forth in SEQ ID Nos.: 2-7, respectively.

Methods are provided of treating or reducing the severity of aortic valve sclerosis and/or aortic stenosis including administering to the subject an effective amount of an antibody or antibody fragment that binds a fragment set forth in SEQ ID No.: 1 of apoB100, and the antibody contains a variable heavy region (V_{H}) as set forth in SEQ ID No.: 8 and a variable light region (V_{L}) containing LCDR1, LCDR2 and LCDR3 as set forth in SEQ ID Nos.: 5-7, respectively. Further aspects of the methods include identifying or selecting a subject with an elevated level of Lp(a) and/or exhibiting symptoms of aortic valve sclerosis and/or aortic stenosis, then administering to the subject the effective amount of the antibody or antibody fragment. Additional aspects further include quantifying the level of Lp(a) and/or symptoms of aortic valve sclerosis or aortic stenosis after the administration, and if the level of Lp(a) or the symptoms persist, continue administering the antibody or antibody fragment.

A further aspect provides that the method of treating or reducing the severity of aortic valve sclerosis and/or aortic stenosis includes administering to the subject an effective amount of an antibody or antibody fragment that binds a fragment set forth in SEQ ID No.: 1 of apoB100, and the antibody contains a variable light region (V_{L}) of SEQ ID No.: 9 and a variable heavy region (V_{H}) that contains HCDR1, HCDR2 and HCDR3 as set forth in SEQ ID Nos.: 2-4, respectively. Further aspects of the methods include identifying or selecting a subject with an elevated level of Lp(a) and/or exhibiting symptoms of aortic valve sclerosis and/or aortic stenosis, then administering to the subject the effective amount of the antibody or antibody fragment. Additional aspects further include quantifying the level of Lp(a) and/or symptoms of aortic valve sclerosis or aortic stenosis after the administration, and if the level of Lp(a) or the symptoms persist, continue administering the antibody or antibody fragment.

Yet another aspect of the invention provides that the method of treating, reducing the severity of aortic valve sclerosis and/or aortic stenosis, and/or reducing the likelihood of aortic valve sclerosis and/or aortic stenosis in a subject, includes administering to the subject an effective amount of an antibody or antibody fragment that binds a fragment set forth in SEQ ID No.: 1 of apoB100, and the antibody contains a variable heavy region (V_{H}) of SEQ ID No.: 8 and a variable light region (V_{L}) of SEQ ID No.: 9. Further aspects of the treatment methods include identifying or selecting a subject with an elevated level of Lp(a) and/or exhibiting symptoms of aortic valve sclerosis and/or aortic stenosis, then administering to the subject the effective amount of the antibody or antibody fragment. Additional aspects of any of the methods further include quantifying the level of Lp(a) and/or symptoms of aortic valve sclerosis or aortic stenosis after the administration, and if the level of Lp(a) or the symptoms persist or appear, continue administering the antibody or antibody fragment.

Methods are provided of treating, reducing the severity of aortic valve sclerosis and/or aortic stenosis, and/or reducing the likelihood of aortic valve sclerosis and/or aortic stenosis, or lowering the level of Lp(a) in a subject, including administering to the subject an effective amount of an antibody or antibody fragment that binds a fragment set forth in SEQ ID No.: 1 of apoB100, and the antibody contains a variable heavy region (V_{H}) as set forth in SEQ ID No.: 8 and a variable light region (V_{L}) containing LCDR1, LCDR2 and LCDR3 as set forth in SEQ ID Nos.: 5-7, respectively. Further aspects of the treatment methods include identifying or selecting a subject with an elevated level of Lp(a) and/or exhibiting symptoms of aortic valve sclerosis and/or aortic stenosis, then administering to the subject the effective amount of the antibody or antibody fragment. Additional aspects of any of these methods further include quantifying the level of Lp(a) and/or symptoms of aortic valve sclerosis or aortic stenosis after the administration, and if the level of Lp(a) or the symptoms persist or appear, continue administering the antibody or antibody fragment.

A further aspect provides that the method of treating, reducing the severity of aortic valve sclerosis and/or aortic stenosis, and/or reducing the likelihood of aortic valve sclerosis and/or aortic stenosis, in a subject includes administering to the subject an effective amount of an antibody or antibody fragment that binds a fragment set forth in SEQ ID No.: 1 of apoB100, and the antibody contains a variable light region (V_{L}) of SEQ ID No.: 9 and a variable heavy region (V_{H}) that contains HCDR1, HCDR2 and HCDR3 as set forth in SEQ ID Nos.: 2-4, respectively. Further aspects of the treatment methods include identifying or selecting a subject with an elevated level of Lp(a) and/or exhibiting symptoms of aortic valve sclerosis and/or aortic stenosis, then administering to the subject the effective amount of the antibody or antibody fragment. Additional aspects of any of the methods further include quantifying the level of Lp(a) and/or symptoms of aortic valve sclerosis or aortic stenosis after the administration, and if the level of Lp(a) or the symptoms persist or appear, continue administering the antibody or antibody fragment.

Yet another aspect of the invention provides that the method of treating, reducing the severity of aortic valve sclerosis and/or aortic stenosis, and/or reducing the likelihood of having aortic valve sclerosis and/or aortic stenosis in a subject, includes administering to the subject an effective amount of an antibody or antibody fragment that binds a fragment set forth in SEQ ID No.: 1 of apoB100, and the antibody contains a variable heavy region (V_{H}) of SEQ ID No.: 8 and a variable light region (V_{L}) of SEQ ID No.: 9. Further aspects of the treatment methods include identifying or selecting a subject with an elevated level of Lp(a) and/or exhibiting symptoms of aortic valve sclerosis and/or aortic stenosis, then administering to the subject the effective amount of the antibody or antibody fragment. Additional aspects of any of the methods further include quantifying the level of Lp(a) and/or symptoms of aortic valve sclerosis or aortic stenosis after the administration, and if the level of Lp(a) or the symptoms persist or appear, continue administering the antibody or antibody fragment.

Methods are also provided of treating or reducing the severity or likelihood of aortic valve sclerosis and/or aortic stenosis, and/or lowering the level of Lp(a), in a subject, which includes administering to the subject an effective amount of an antibody or antibody fragment that binds a fragment set forth in SEQ ID No.: 1 of apoB100, and the antibody contains a heavy chain of SEQ ID No.: 10 and a light chain containing LCDR1, LCDR2 and LCDR3 as set forth in SEQ ID Nos.: 5-7, respectively. Further aspects of the treatment methods include identifying or selecting a subject with an elevated level of Lp(a) and/or exhibiting symptoms of aortic valve sclerosis and/or aortic stenosis, then administering to the subject the effective amount of the antibody or antibody fragment. Additional aspects of any of the methods further include quantifying the level of Lp(a) and/or symptoms of aortic valve sclerosis or aortic stenosis after the administration, and if the level of Lp(a) or the symptoms persist or appear, continue administering the antibody or antibody fragment.

A further aspect of the embodiment provides that the method includes administering to the subject an effective amount of an antibody or antibody fragment that binds a fragment set forth in SEQ ID No.: 1 of apoB100, and the antibody contains a heavy chain of SEQ ID No.: 10 and a light chain that contains a variable light region (V_{L}) of SEQ ID No.: 9. Another aspect of the invention provides the method includes administering to the subject an effective amount of an antibody or antibody fragment that binds a fragment set forth in SEQ ID No.: 1 of apoB100, and the antibody contains a light chain of SEQ ID No.: 11 and a heavy chain that contains HCDR1, HCDR2 and HCDR3 as set forth in SEQ ID Nos.: 2-4, respectively. Further aspects of the treatment methods include identifying or selecting a subject with an elevated level of Lp(a) and/or exhibiting symptoms of aortic valve sclerosis and/or aortic stenosis, then administering to the subject the effective amount of the antibody or antibody fragment. Additional aspects of any of the methods further include quantifying the level of Lp(a) and/or symptoms of aortic valve sclerosis or aortic stenosis after the administration, and if the level of Lp(a) or the symptoms persist or appear, continue administering the antibody or antibody fragment.

Yet another aspect provides the method includes administering to the subject an effective amount of an antibody or antibody fragment that binds a fragment set forth in SEQ ID No.: 1 of apoB100, and the antibody contains a light chain of SEQ ID No.: 11 and a heavy chain that contains a variable heavy region (V_{H}) of SEQ ID No.: 8. Further aspects of the treatment methods include identifying or selecting a subject with an elevated level of Lp(a) and/or exhibiting symptoms of aortic valve sclerosis and/or aortic stenosis, then administering to the subject the effective amount of the antibody or antibody fragment. Additional aspects of any of the methods further include quantifying the level of Lp(a) and/or symptoms of aortic valve sclerosis or aortic stenosis after the administration, and if the level of Lp(a) or the symptoms persist or appear, continue administering the antibody or antibody fragment.

Alternatively, the method includes administering to the subject an effective amount of an antibody or antibody fragment that binds a fragment set forth in SEQ ID No.: 1 of apoB100, and the antibody contains a heavy chain of SEQ ID No.: 10 and a light chain of SEQ ID No.: 11. Further aspects of the treatment methods include identifying or selecting a subject with an elevated level of Lp(a) and/or exhibiting symptoms of aortic valve sclerosis and/or aortic stenosis, then administering to the subject the effective amount of the antibody or antibody fragment. Additional aspects of any of the methods further include quantifying the level of Lp(a) and/or symptoms of aortic valve sclerosis or aortic stenosis after the administration, and if the level of Lp(a) or the symptoms persist or appear, continue administering the antibody or antibody fragment.

### Patient Selection

Embodiments provide that the methods for treating a cardiovascular disease (e.g., aortic valve stenosis or aortic valve sclerosis) by inhibiting the formation of Lp(a) includes administering the pharmaceutical composition to a subject diagnosed with or showing symptoms of the disease.

Further aspects of any of the treatment methods include identifying or selecting a subject with an elevated level of Lp(a) and/or exhibiting symptoms of aortic valve sclerosis and/or aortic stenosis, then administering to the subject the effective amount of the antibody or antibody fragment.

Additional aspects of any of the treatment or reducing likelihood of development methods further include quantifying the level of Lp(a) and/or symptoms of aortic valve sclerosis or aortic stenosis after the administration, and if the level of Lp(a) or the symptoms persist or appear, continue administering the antibody or antibody fragment.

In some aspects, a reduced amount or level of Lp(a) after the administration of the antibody or antibody fragment is relative to the amount of the same subject before the administration, or relative to the amount from subjects not having aortic valve sclerosis or aortic stenosis or having been successfully treated from aortic valve sclerosis or aortic stenosis.

In other aspects, an elevated amount or level of Lp(a) is relative to the amount or level of subject not having aortic valve sclerosis or aortic stenosis or having been successfully treated from aortic valve sclerosis or aortic stenosis.

### Combination Therapy

Some embodiments provide that the antibody or antibody fragment for inhibiting or reducing the formation of Lp(a) and/or in the treatment of aortic valve stenosis or aortic valve sclerosis described herein, is used in combination with an existing treatment for coronary artery diseases (CADs). For example, a method for treating a subject with aortic valve stenosis or aortic valve sclerosis includes administering an effective amount of an anti-ApoB100 and an effective amount of one or more of statins, anti-platelet agents, beta blockers, angiotensin-converting enzyme (ACE) inhibitors, and calcium channel blockers, optionally in addition to surgery (such as angioplasty and stent placement, fibrinolytic therapy, percutaneous coronary intervention (PCI), coronary artery bypass grafting (CABG), carotid endarterectomy).

The pharmaceutical compositions including the antibody or antibody fragment, optionally further including with one, two, three or more existing treatments for CADs for inhibiting or reducing the formation of Lp(a) and in the treatment of aortic valve stenosis or aortic valve sclerosis may be provided with commonly used adjuvants to enhance absorption of the antibody or mixture of antibodies. In various embodiments, the compositions according to the invention may be formulated for delivery via any route of administration. “Route of administration” may refer to any administration pathway known in the art, including but not limited to aerosol, nasal, oral, transmucosal, transdermal, parenteral or enteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection.

### Dosage

In one embodiment, an effective amount of the antibody or antibody variant that binds SEQ ID NO:1 fragment of ApoB100 results in plasma concentration of the antibody of at least 4 μg/mL, preferably at least 12 μg/mL. Some embodiments provide the composition for inhibiting or reducing Lp(a) is orticumab of at least about 8 mg/kg of a patient (e.g., 664 mg for an averaged human patient of 83 kg). Some embodiments provide that the composition for inhibiting or reducing Lp(a) is orticumab of between 5 mg/kg of a patient (e.g., 415 mg for an averaged human patient of 83 kg) and 8 mg/kg. Some embodiment provides administering orticumab at a monthly dosing regimen at the above-mentioned dosage.

Other embodiments provide the anti-ApoB100 is administered weekly at no less than 2 mg/kg/week (166 mg for an averaged human patient of 83 kg); preferably, 4 mg/kg/week (332 mg for an averaged human patient of 83 kg). In another aspect, the composition of an anti-ApoB100 or anti-apo(a) antibody is administered biweekly at >2.5 mg/kg/two weeks (e.g., 208 mg for an averaged human patient of 83 kg). In yet another aspect, the composition of an anti-ApoB100 or anti-apo(a) antibody is administered monthly at about 6 mg/kg/month (e.g., about 498 mg for an averaged human patient of 83 kg). For example, the monthly dosing may be carried out for 12 months or 3 months.

Some embodiments provide an effective amount of the composition includes at least an initial dose of the antibody of approximately 800-900 mg, 900-1000 mg, 1000-1100 mg, 1100-1200 mg, 1200-1300 mg, 1300-1400 mg, 1400-1500 mg, or 1500-1600 mg. In some aspects, the effective amount in the method described herein includes an initial dose of orticumab of approximately 1000-1500 mg, followed by subsequent doses of the antibody at 700-900 mg administered weekly for 2, 3, 4 or 5 weeks and/or even administered monthly for 1, 2 or 3 months.

Another exemplary embodiment provides step-wise escalating doses of the antibody or antibody fragment that binds SEQ ID NO:1 fragment of ApoB100. In this embodiment, an exemplary (starting) dose of a single-dose administration of an antibody against ApoB100 or apo(a) is between 0.005 and 0.01 mg/kg (e.g., intravenously); and other exemplary dosage levels to be administered in the single-dose administration are between 0.01 and 0.15, between 0.15 and 0.75, between 0.75 and 2.5, between 2.5 and 7.5, and between 7.5 and 30 mg/kg (e.g., intravenously). For example, a starting dose of an antibody against ApoB100 or apo(a) in a single-dose intravenous administration is 0.007 mg/kg; and other exemplary dosages can be 0.05, 0.25, 1.25, 5.0 or 15.0 mg/kg in subsequent single-dose intravenous administration. In another embodiment, a single-dose subcutaneous administration of an antibody against ApoB100 or apo(a) is between 0.5 and 5 mg/kg, and a multiple-dose subcutaneous administration is also between 0.5 and 5 mg/kg. For example, an antibody against ApoB100 or apo(a) at 1.25 mg/kg is administered subcutaneously. In various embodiments, the dosage is administered within a specified hour range of the day in each administration, and each dose in a multiple-dose treatment (e.g., 4 doses, 3 doses, 5 doses, or 6 doses) is administered at weekly intervals with a time window of ±1 day. In another example, of an antibody against ApoB100 or apo(a) is administered at between 300 mg and 450 mg (e.g., 360 mg) to a human subject, optionally followed by another dose between 300 mg and 450 mg (e.g., 360 mg) to the human subject where the second dose is at least 70 days (up to 91 days) apart from the first dose. The antibody against ApoB100 or apo(a) may be formulated at a concentration of 100-170 mg/mL (e.g., 150 mg/mL) and for use in subcutaneous administration without further dilution, or diluted to a large volume for intravenous infusion.

Further embodiments include administering to a subject an effective amount of an antibody or antibody fragment that binds SEQ ID No.:1 and having a sequence of one or more of SEQ ID Nos: 2-11, which is in the range of about 10-50 μg/period, 50-100 μg/period, 100-150 μg/period, 150-200 μg/period, 100-200 μg/period, 200-300 μg/period, 300-400 μg/period, 400-500 μg/period, 500-600 μg/period, 600-700 μg/period, 700-800 μg/period, 800-900 μg/period, 900-1000 μg/period, 1000-1100 μg/period, 1100-1200 μg/period, 1200-1300 μg/period, 1300-1400 μg/period, 1400-1500 μg/period, 1500-1600 μg/period, 1600-1700 μg/period, 1700-1800 μg/period, 1800-1900 μg/period, 1900-2000 μg/period, 2000-2100 μg/period, 2100-2200 μg/period, 2200-2300 μg/period, 2300-2400 μg/period, 2400-2500 μg/period, 2500-2600 μg/period, 2600-2700 μg/period, 2700-2800 μg/period, 2800-2900 μg/period or 2900-3000 μg/period. A period is a day, a week, a month, or another length of time. One aspect is the antibody (e.g., orticumab) is administered at a weekly, biweekly or monthly frequency of any of above-mentioned dosage per period.

In some embodiments, the methods include administering an inhibitor of oxidized LDL (e.g., orticumab) to the subject for 1-5 days, 1-5 weeks, 1-5 months, or 1-5 years. For example, the antibody is administered to the subject in 3, 4, 5, 6, 7, 8, 9, 10, 11, 12, 13, 14, 15, 16, 17, 18, 19 or 20 doses, each dose separated by at least 3 days, 5 days, one week, two weeks, one month, two months, or a combination thereof. In other embodiments, the second dose is administered about 2-3 weeks, or about 3 weeks after the first dose and the third dose is administered about 5-6 weeks or about 6 weeks after the first dose, etc. In another embodiment, the second dose is administered about 2-3 months, about 2 months, about 3 months or about 4 months after the first dose and the third dose is administered about 4-6 months, about 5-6 months, about 5 months or about 6 months after the first dose.

### Pharmaceutical Composition or Medicaments

In various embodiments, the present invention provides a pharmaceutical composition for use with the methods described herein. The pharmaceutical composition includes a composition that inhibits or reduces the formation of Lp(a), such as antibody or antibody fragment against ApoB100, and a pharmaceutically acceptable carrier.

Further embodiments provide that a composition or medicament for use in treating, reducing the severity or likelihood of aortic valve sclerosis or aortic stenosis, and/or lowering the level of Lp(a) in a subject, where the composition of medicament contains an anti-oxLDL antibody that binds to an epitope of SEQ ID No.:1 of ApoB100, as disclosed above, is in an amount of between 300 mg and 400 mg, preferably about 330 mg, per dosage (or vial), optionally with a pharmaceutically acceptable carrier, each (e.g., for a monthly subcutaneous administration to a subject) for 1, 2, 3, 4, 5, 6, 7, 8, 9, 10, 11, 12 months or longer. Other embodiments provide the composition or medicament contains an anti-oxLDL antibody that binds to an epitope of SEQ ID No.:1 of ApoB100, as disclosed above, in an amount of at least 5, 6, 7, or 8 mg orticumab/kg of a patient in one dosage (or vial), and optionally more dosages (or vials) of at least 2 mg/kg/week, at least 2.5 mg/kg/two weeks, or at least 6 mg/kg/month, for 3, 4, 5, 6, 7, 8, 9, 10, 11, 12 months or longer. Further embodiments provide the composition or medicament contains the antibody (such as orticumab) at a concentration of 100-170 mg/mL (e.g., 150 mg/mL) and for use in subcutaneous administration without further dilution, or diluted to a large volume for intravenous infusion.

“Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof. Generally, each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

### Prepare Antibodies in the Methods

In some embodiments, the aforementioned methods involve antibodies that bind to a specific antigen epitope, where the antibodies contain one or more defined sequences. For example, modern recombinant library technology is used to prepare therapeutic antibodies against native ApoB, oxidized ApoB or MBA-modified ApoB. While murine hybridomas cells produce large amounts of identical antibodies, these non-human antibodies are recognized by human body as foreign, and as a consequence, their efficacy and plasma half-lives are decreased in addition to eliciting allergic reactions. To solve this problem, one approach is to make chimeric antibodies where the murine variable domains of the antibody are transferred to human constant regions resulting in an antibody that is mainly human. A further refinement of this approach is to develop humanized antibodies where the regions of the murine antibody that contacted the antigen, the so called Complementarity Determining Regions (CDRs) are transferred to a human antibody framework, resulting in a humanized antibody. Another approach is to produce completely human antibodies using recombinant technologies, which does not rely on immunization of animals to generate the specific antibody. Instead recombinant libraries comprise a huge number of pre-made antibody variants and it is likely that a library will have at least one antibody specific for any antigen. A phage display system may be used where antibody fragments are expressed, displayed, as fusions with phage coat proteins on the surface of filamentous phage particles, while the phage display system simultaneously carries the genetic information encoding the displayed molecule. Phage displaying antibody fragments specific for a particular antigen may be selected through binding to the antigen in question. Isolated phage may then be amplified and the gene encoding the selected antibody variable domains may optionally be transferred to other antibody formats as e.g. full length immunoglobulin and expressed in high amounts using appropriate vectors and host cells well known in the art. The format of displayed antibody specificities on phage particles may differ. The most commonly used formats are Fab and single chain (scFv) both containing the variable antigen binding domains of antibodies. The single chain format is composed of a variable heavy domain (V_{H}) linked to a variable light domain (V_{L}) via a flexible linker. Before use as analytical reagents, or therapeutic agents, the displayed antibody specificity is transferred to a soluble format, e.g., Fab or scFv, and analyzed as such. In later steps the antibody fragment identified to have desirable characteristics may be transferred into yet other formats such as full length antibodies.

Antibody Production Using Hybridomas

The cell fusions are accomplished by standard procedures well known to those skilled in the field of immunology. Fusion partner cell lines and methods for fusing and selecting hybridomas and screening for mAbs are well known in the art. See, e.g., Ausubel infra, Harlow infra, and Colligan infra, the contents of which references are incorporated entirely herein by reference.

An anti-ApoB100 antibody, or an anti-apo(a) antibody, can be produced in large quantities by injecting hybridoma or transfectoma cells secreting the antibody into the peritoneal cavity of mice and, after appropriate time, harvesting the ascites fluid which contains a high titer of the mAb, and isolating the mAb therefrom. For such in vivo production of the mAb with a non-murine hybridoma (e.g., rat or human), hybridoma cells are preferably grown in irradiated or athymic nude mice. Alternatively, the antibodies can be produced by culturing hybridoma or transfectoma cells in vitro and isolating secreted mAb from the cell culture medium or recombinantly, in eukaryotic or prokaryotic cells.

Recombinant Expression of Anti-ApoB100

Recombinant murine or chimeric murine-human or human-human antibodies that bind ApoB100 can be provided according to the present invention using known techniques based on the teaching provided herein. See, e.g., Ausubel et al., eds. Current Protocols in Molecular Biology, Wiley Interscience, N.Y. (1987, 1992, 1993); and Sambrook et al. Molecular Cloning: A Laboratory Manual, Cold Spring Harbor Laboratory Press (1989).

The DNA encoding an anti-ApoB100 antibody, or an anti-apo(a) antibody can be genomic DNA or cDNA which encodes at least one of the heavy chain constant region (Hc), the heavy chain variable region (Hc), the light chain variable region (Lv) and the light chain constant regions (Lc). A convenient alternative to the use of chromosomal gene fragments as the source of DNA encoding the murine V region antigen-binding segment is the use of cDNA for the construction of chimeric immunoglobulin genes, e.g., as reported by Liu et al. (Proc. Natl. Acad. Sci., USA 84:3439 (1987) and J. Immunology 139:3521 (1987). The use of cDNA requires that gene expression elements appropriate for the host cell be combined with the gene in order to achieve synthesis of the desired protein. The use of cDNA sequences is advantageous over genomic sequences (which contain introns), in that cDNA sequences can be expressed in bacteria or other hosts which lack appropriate RNA splicing systems.

### Screening Assays

Various embodiments provide a method for identifying a molecule or compound that reduces the binding between apolipoprotein (a) and low-density lipoprotein (LDL), and inhibits the formation of lipoprotein(a) (Lp(a)). The method includes contacting a molecule or compound of interest with a mixture of LDL and apolipoprotein (a); determining whether the contact between the molecule or compound of interest and the mixture results in a decrease in the binding between apolipoprotein (a) and LDL, a decrease in the amount of Lp(a), or both, compared to that in a mixture without the molecule or compound of interest, wherein a decrease in the binding between apolipoprotein (a) and LDL or a decrease in the amount of Lp(a) indicates that the molecule or compound of interest reduces the binding between apolipoprotein (a) and LDL, and inhibits the formation or reduces the amount of Lp(a).

In some embodiments, the molecule or compound is selected from the group consisting of a small molecule, a polypeptide, a peptide, an antibody or a fragment thereof and a nucleic acid molecule. In some embodiments, the molecule or compound of interest binds to ApoB100.

In further embodiments, the molecule or compound of interest reduces the likelihood or progression of aortic valve sclerosis or aortic stenosis.

Exemplary assays for methods of identifying a compound or molecule of interest includes western blot analysis or mass spectrometry to separate and quantify Lp(a) in comparison to its constituent (apo(a) and/or LDL) based on the size of these biomolecules from a sample, binding assays to quantify the amount of the constituent of Lp(a), fluorescently labeling the constituents and quantifying the quenching or appearance of fluorescent signals as indication of physical proximity of the constituents when bound. Further details on the exemplary assays can be seen in Examples below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1: Antibodies Blocking the Association Between ApoB100 and Apo(a), Thereby Inhibiting the Assembly of Lp(a)

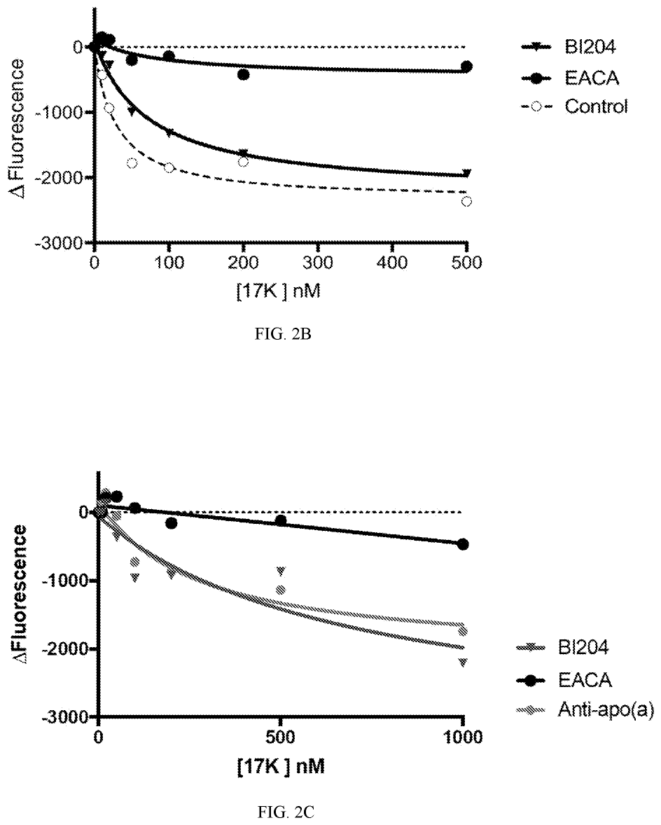

FIG. 2 shows using antibodies capable of binding the binding sites on the Apolipoprotein B100 molecule, the assembly of Apolipoprotein B100 with apolipoprotein (a), thereby the formation of Lp(a), was prevented. ECAC refers to epsilon-aminocaproic acid, which is a derivative and analogue of the amino acid lysine, making it an effective inhibitor for proteins that bind that particular residue. Orticumab (BI204) showed a similar inhibition effect on the formation of Lp(a) to a sheep polyclonal anti-apo(a) antibody (denoted “anti-apo(a)”).

As such, Applicant demonstrated that orticumab could prevent the binding of ApoB100 and therefore the formation of Lp(a), which is believed to have significant diagnostic and therapeutic implications in the pathophysiologic mechanisms and the clinical treatment of diseases.

### Example 2: Prevention of AVS Progression by Blocking Lp(a) Formation with Orticumab

A phase 2 clinical study with a plan as follows: N=100 patients that are early stage AVS subjects younger than 58 years old with elevated Lp(a) in serum and mild AVS (defined by echocardiography). Lp(a) is measured as the total quantity of LDL-c. Endpoints are to assess the slowing of the progression of AVS, measured by echocardiography.

### Example 3: Inhibition of Non-Covalent and Covalent Binding Between Apo(a) and LDL, Thereby Inhibiting the Assembly of Lp(a)

Inhibition of non-covalent and covalent binding between apo(a) and LDL were assessed by fluorescence and Western blot analysis, respectively. Orticumab was demonstrated very effective in inhibiting non-covalent binding. However fluorescence interference by antibodies made the effect on non-covalent binding inconclusive. Covalent binding was inhibited by orticumab at all concentrations (0.01, 0.1, 1, and 10 μM).

Non-covalent interaction between apo(a) and LDL:

LDL was purified from the plasma of a healthy donor using sequential density gradient ultracentrifugation and labeled using the thiol-directed probe 5′-iodoacetamidofluorescein. Recombinant apo(a) variants (r-apo(a): 17K and 17KALBS7,8) were purified from a serum-free conditioned medium that was harvested from stably-expressing HEK293 cell lines. The lysine analogue, ε-aminocaproic acid (EACA), commercially available sheep polyclonal anti-apo(a) antibody, and commercially available goat polyclonal anti-apoB-100 antibody were used as positive controls. Titrations were conducted at room temperature in 96-well round-bottom white plates, and proteins were diluted in 20 mM HEPES pH 7.4, 150 mM NaCl, 0.01% Tween 20 (HBST). Measurements of fluorescence were performed using SPECTRAMAX® M2e (Molecular Devices); excitation and emission wavelengths were 495 nm and 530 nm, respectively, with a 535-nm cut-off filter placed in the emission beam.

Covalent interaction between apo(a) and LDL:

Purified LDL (100 nM) and 17K apo(a) (5 nM) were incubated with conditioned serum-free medium (OptiMEM; Gibco) from HEK293 cells in the presence of 0, 0.01, 0.1, 1.0, and 10 μM of orticumab at 37° C. The reactions were mixed thoroughly every hour and stopped after 4 or 8 hrs by adding 4×SDS-PAGE sample buffer. Samples were then boiled for 5 min at 95° C. and subjected to SDS-PAGE on 6% polyacrylamide gel. After electrophoresis, separated proteins were electroblotted onto a PVDF membrane, blocked, and incubated with a monoclonal antibody (A5) specific for apo(a). HRP-conjugated goat anti-mouse IgG secondary antibody was used to develop a signal in the presence of a chemiluminescence reagent and images were captured using a ChemiDoc system (Bio-Rad). Band intensity (BI) of western-blots were quantified using IMAGELAB software for recombinant Lp(a) formation using the following equation: % r-Lp(a)=BI(Lp(a))/[BI(Lp(a))+BI(apo(a))]. The means±SD of three independent experiments are presented in FIG. 10. A Two-Way ANOVA with Tukey post-hoc test was used to analyze the data in GraphPad Prism 7.0.

Non-covalent interaction between apo(a) and LDL:

Inhibition of non-covalent binding was measured in a fluorescence assay using fluorescently-labeled LDL (Flu-LDL). In this assay, fluorescence is quenched when non-covalent binding occurs between apo(a) and LDL. Flu-LDL (50 nM) was combined with 17K r-apo(a) at 0, 10, 50, 100, 200, 500, and 1000 nM in the presence of orticumab (1000 nM). The lysine analog EACA, which inhibits non-covalent binding, was used at a concentration of 100 mM. FIG. 2B shows orticumab inhibited the binding, as reflected by the shift of the curve to the right and the increased apparent Kd. In FIG. 2C, a polyclonal anti-apo(a) antibody, which inhibits non-covalent binding, was also used as a positive control; the data in this figure show that inhibition of non-covalent binding by orticumab is on par with the anti-apo(a) antibody. The fluorescence observed in the absence of antibody (“control” in FIG. 2B), when quenching of the Flu-LDL fluorescence by apo(a) is lowest, was used as a correction factor in determining the fluorescence in the presence of antibodies and EACA. The lines in the graph depict non-linear regression of the data fit to a rectangular hyperbola.

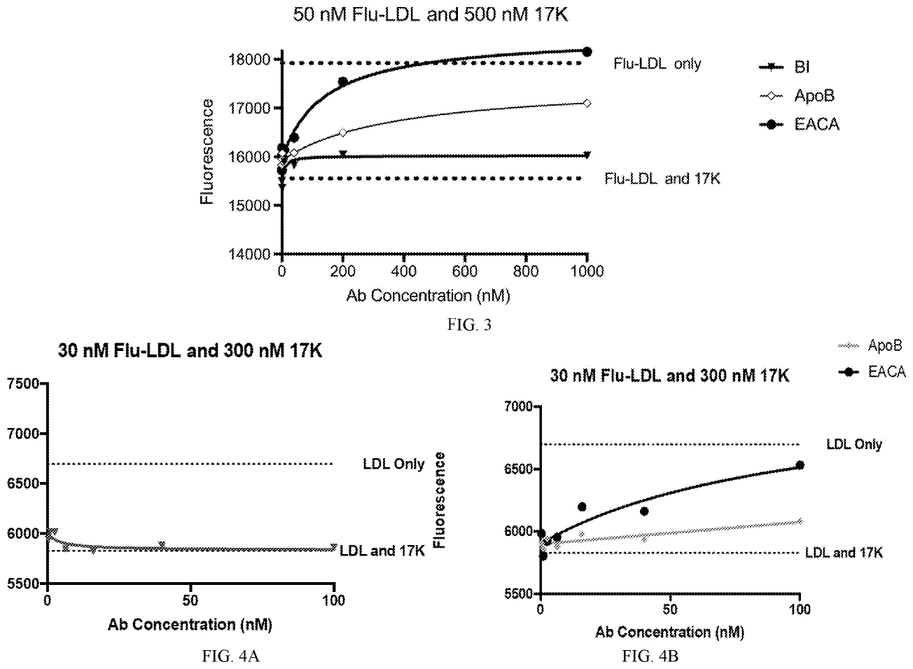

Next, non-covalent binding between apo(a) and LDL was assessed using titrated concentrations of orticumab and a fixed concentration of 17K r-apo(a) in 10-fold molar excess over Flu-LDL. A commercially available polyclonal anti-apoB antibody, which interferes with the non-covalent interaction, was used as a positive control. In this experiment, orticumab was able to alleviate the quenching of Flu-LDL by 17K apo(a) (FIG. 3). However, the inhibitory effect of orticumab was much lower than that of the polyclonal anti-apoB antibody or EACA.

To better capture the response at concentrations between 0-200 nM, lower concentrations of orticumab, Flu-LDL, and 17K r-apo(a) were used (FIG. 4A). The concentration of 17K r-apo(a) and LDL remained fixed, with 17K r-apo(a) in 10-fold molar excess over Flu-LDL. In comparison with EACA or polyclonal anti-ApoB antibody (denoted as “ApoB”) (FIG. 4B), orticumab was not able to de-quench (inhibit) the fluorescence, even after lowering the level of total Flu-LDL and apo(a). However, the presence of orticumab interfered with fluorescence even at concentrations as low as 0.4 nM of orticumab (FIG. 4A).

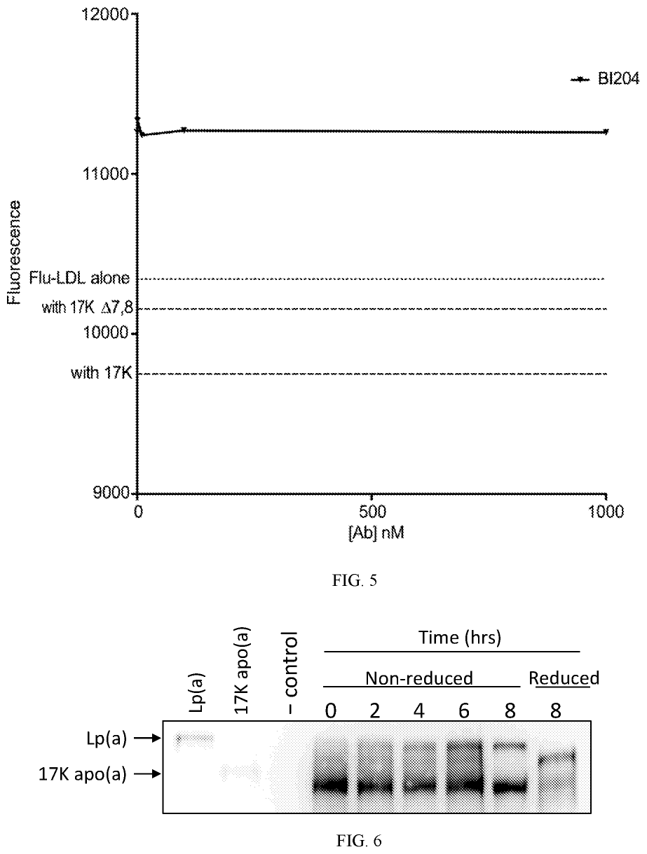

The interference with LDL fluorescence in response to orticumab was explored further, using Flu-LDL alone or in the presence of orticumab, 17K r-apo(a), and 17K Δ7,8 apo(a). In comparison with Flu-LDL alone, orticumab increased the fluorescence of Flu-LDL to a higher level (FIG. 5). This increase in fluorescence may affect our interpretation of the previous results.

Covalent interaction between apo(a) and LDL

Covalent binding between apo(a) and LDL was assessed over time by appearance of Lp(a) protein on a Western blot. Using serum-free HEK293 cell-conditioned medium, Lp(a) formation was evaluated after incubation of 17K r-apo(a) with LDL from 0-8 hours; isolated Lp(a), 17K apo(a) alone, and media alone were used as controls. The data show that Lp(a) formation begins to occur at 4 hours with maximum formation occurring by 8 hours (FIG. 6).

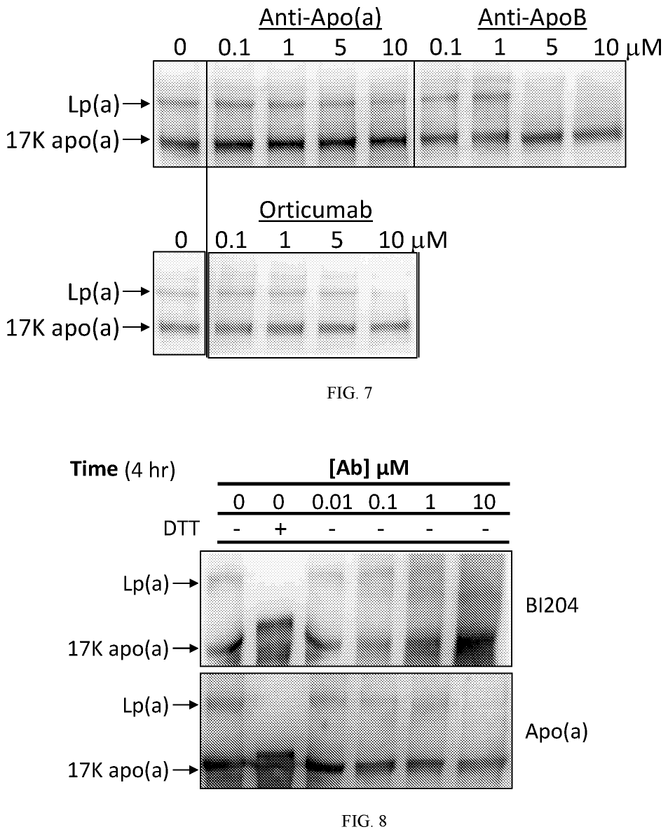

Next, the effect of orticumab on inhibition of covalent binding between apo(a) and LDL was assessed by Western blot analysis. 17K r-apo(a) (5 nM) was incubated with LDL (100 nM) in serum free HEK293 cell-conditioned medium for 4 hours at 37° C. in the presence of orticumab at 0.1, 1, 5, and 10 μM. Disappearance of the Lp(a) band was observed with 10 μM orticumab (FIG. 7).

The Western blot analysis was repeated with orticumab at 0.01 μM, 0.1 μM, 1 μM or 10 μM and including only anti-apo(a) as a control. In comparison to the anti-apo(a) antibody, only orticumab behaves similarly in reducing covalent Lp(a) (FIG. 8).

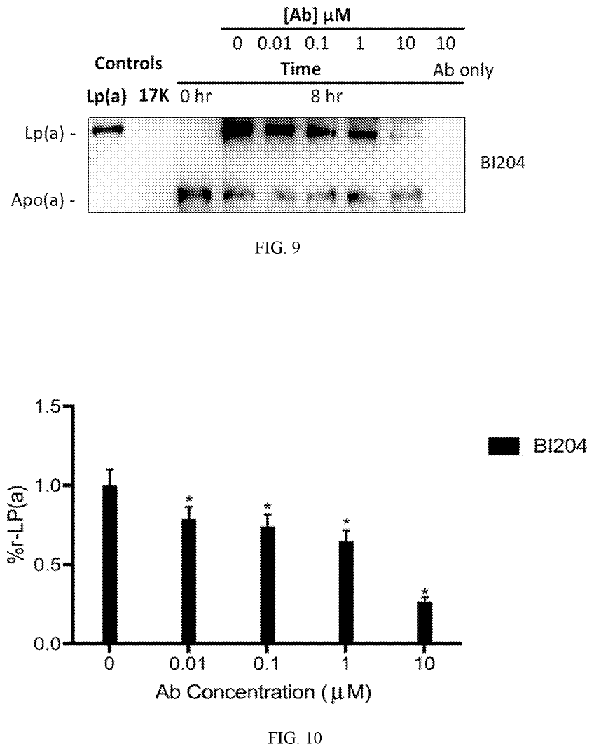

To determine whether inhibition of Lp(a) formation occurs at a later time point when Lp(a) formation appears to be maximal, the Western blot experiments were repeated after a 0- and 8-hour incubation of 5 nM 17K r-apo(a) and 100 nM LDL in the presence of orticumab at 0.01, 0.1, 1, and 10 μM in serum free HEK293 cell-conditioned medium (FIG. 9). The results were then quantified (FIG. 10) and analyzed. The data show that orticumab significantly inhibited Lp(a) formation at the highest concentration used (10 μM) and at the lower concentrations (0.01, 0.1, and 1 μM). At 10 μM, the percent inhibition of Lp(a) formation was 73.46% with orticumab (p<0.0001 for each). At 1 Lp(a) formation was inhibited by 34.99% with orticumab, respectively (p<0.0001 for each); at 0.1 μM, 25.8% with orticumab (p=0.0027); and at 0.01 μM, 21.37% with orticumab (p=0.0177).

Lp(a) is an independent and causal risk factor for cardiovascular diseases and the single most prevalent inherited risk factor. Lp(a) assembly occurs by the interaction of apo(a) and apoB in a two-step process. In the first step, non-covalent interaction occurs between kringles IV-7 and -8 in apo(a) and the N-terminus of apoB; this facilitates the second step for covalent assembly by the formation of a disulfide bond between kringle IV-9 and apoB. Interfering with the non-covalent and covalent Lp(a) assembly (i.e., production) has clinical significance for the lowering of Lp(a) levels.

In this study, we tested the ability of orticumab, which is raised against human apoB-100 epitope residues 661-680, to block the non-covalent and covalent assembly of Lp(a) in vitro.

In the non-covalent assay, we assessed the ability of the antibody to restore the fluorescence of Flu-LDL that is quenched by 17K apo(a). First, we found orticumab (at 1000 nM) interfered with the apo(a) and Flu-LDL interaction in a 17K apo(a) titration, showing effects similar to an anti-apo(a) antibody. Next, orticumab titrated to generate dose-response curves. The maximum response was seen at 1000 nM with orticumab. At lower concentrations (≤100 nM), orticumab showed no effect on de-quenching the fluorescence. Finally, as a quality control step, orticumab was run with Flu-LDL alone. This experiment showed an interference of Flu-LDL in the presence of orticumab, as demonstrated by an enhanced fluorescent signal. Therefore, the fluorescence interference makes the validation of these antibodies in our system for non-covalent assembly more complex, especially when IC50 values are to be measured.

Covalent assembly between purified 17-kringle apo(a) (5 nM) and purified human LDL (100 nM) occurs in vitro, forming Lp(a) which can be resolved using SDS-PAGE and Western blotting. Using this assay, we show that Lp(a) formation occurs over time with the first prominent bands appearing after 4 hours of incubation. Therefore, at 4 hours, inhibition of Lp(a) assembly was evaluated using orticumab at 0, 0.01, 0.1, 1.0, and 10 μM concentration. Orticumab was as effective as the anti-apoB or anti-apo(a) control antibodies in inhibiting Lp(a) assembly. After 8 hours, we were able to demonstrate that orticumab decreased Lp(a) covalent assembly at the lowest concentration tested (0.01 μM). Therefore, orticumab is shown to inhibit the covalent assembly of Lp(a). The present analysis shows that orticumab can act as inhibitors to block Lp(a) assembly in vitro.

To validate a biological function of orticumab on Lp(a) assembly, cell-based and in vivo systems are envisioned. Possible systems include a human hepatoma cell-culture model expressing endogenous apoB100 and ectopic apo(a), or human Lp(a) transgenic mice expressing human apo(a) and human apoB (or primary hepatocytes isolated from these animals).

Currently there are no drugs approved for the treatment of AVS. Although antisense gene therapy is being investigated to knock out apo(a), there are concerns regarding neoplasm formation with the antisense gene therapy.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

The term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

What is claimed is:

1. A method for treating, reducing the severity of, slowing progression of or reducing the likelihood of aortic valve sclerosis or aortic stenosis in a subject in need thereof, comprising:
administering to the subject an effective amount of a pharmaceutical composition comprising an antibody or antibody fragment capable of binding a fragment of apolipoprotein B100 (ApoB100),
wherein the fragment of ApoB100 comprises an amino acid sequence of SEQ ID No.: 1 or an active site thereof, and the antibody or the antibody fragment comprises one, two or three heavy chain complementarity determining regions (HCDRs) selected from the group consisting of HCDR 1 (HCDR1), HCDR 2 (HCDR2) and HCDR 3 (HCDR3) sequences of SEQ ID Nos: 2, 3 and 4, respectively, and one, two or three light chain complementarity determining regions (LCDRs) selected from the group consisting of LCDR 1 (LCDR1), LCDR 2 (LCDR2) and LCDR 3 (LCDR3) sequences of SEQ ID Nos: 5, 6 and 7, respectively.

2. The method of claim 1, further comprising selecting a subject with an elevated level of Lp(a) prior to administering the effective amount of the pharmaceutical composition.

3. The method of claim 1, further comprising measuring the level of Lp(a) in the subject after the administration.

4. The method of claim 1, wherein the subject is determined to have a reduced amount of lipoprotein(a) (Lp(a)) after the administration.

5. The method of claim 1, wherein the fragment of ApoB100 is an aldehyde derivative.

6. The method of claim 1, wherein the antibody comprises a variable heavy region (V_{H}) of SEQ ID No.: 8, a variable light region (V_{L}) of SEQ ID No.: 9, or both.

7. The method of claim 1, wherein the antibody comprises heavy chain of SEQ ID No.: 10, a light chain of SEQ ID No.: 11, or both.

8. The method of claim 1, wherein the antibody is orticumab.

9. The method of claim 1, wherein the pharmaceutical composition further comprises a pharmaceutically acceptable carrier or excipient.

10. The method of claim 1, wherein the subject is a human, the antibody is orticumab, and orticumab is administered subcutaneously at a dose of about 330 mg/month for about 3, 4, 5, 6, 7, 8, 9, 10, 11 or 12 months.

11. The method of claim 5, wherein the orticumab is administered at an initial dose of at least 5 mg/kg, optionally followed by a plurality of subsequent doses each in an amount of at least 2 mg/kg/week, at least 2.5 mg/kg/two weeks, or at least 6 mg/kg/month.

12. The method of claim 1, wherein the antibody or antibody fragment is administered at 1-10 μg/kg, 10-100 μg/kg, 100-500 μg/kg, 200-500 μg/kg, 300-500 μg/kg, 400-500 μg/kg, 1-5 mg/kg, 5-10 mg/kg, 10-15 mg/kg, 15-20 mg/kg, 20-25 mg/kg, 25-50 mg/kg, 50-75 mg/kg.

13. A method of reducing the level of lipoprotein(a) (Lp(a)) in a subject, comprising:
administering to the subject an amount of a pharmaceutical composition effective to reduce the level of Lp(a) in the subject,
wherein the pharmaceutical composition comprises an antibody or antibody fragment capable of binding a fragment of apolipoprotein B100 (ApoB100), wherein the fragment of ApoB100 comprises an amino acid sequence of SEQ ID No.: 1 or an active site thereof, and the antibody or the antibody fragment comprises one, two or three heavy chain complementarity determining regions (HCDRs) selected from the group consisting of HCDR 1 (HCDR1), HCDR 2 (HCDR2) and HCDR 3 (HCDR3) sequences of SEQ ID Nos: 2, 3 and 4, respectively, and one, two or three light chain complementarity determining regions (LCDRs) selected from the group consisting of LCDR 1 (LCDR1), LCDR 2 (LCDR2) and LCDR 3 (LCDR3) sequences of SEQ ID Nos: 5, 6 and 7, respectively.

14. The method of claim 13, further comprising measuring the level of Lp(a) in the subject after the administration, and the level of Lp(a) is reduced for at least 20%, 30%, 40%, or 50% compared to the level before the administration.

15. The method of claim 13, wherein the subject is diagnosed with or shows symptoms of a cardiovascular disease.

16. The method of claim 15, wherein the cardiovascular disease comprises calcific aortic valve sclerosis or stenosis, and the subject has a reduced symptom or reduced progression of the calcific aortic valve sclerosis or stenosis compared to that before the administration.

17. The method of claim 13, wherein the antibody comprises a variable heavy region (V_{H}) of SEQ ID No.: 8, a variable light region (V_{L}) of SEQ ID No.: 9, or both.

18. The method of claim 13, wherein the antibody is orticumab, and the orticumab is administered at an initial dose of at least 5 mg/kg, optionally followed by a plurality of subsequent doses each in an amount of at least 2 mg/kg/week, at least 2.5 mg/kg/two weeks, or at least 6 mg/kg/month.

19. The method of claim 11, further comprising selecting a subject with an elevated level of Lp(a) prior to administering the effective amount of the pharmaceutical composition comprising an antibody or antibody fragment capable of binding a fragment of ApoB100.

20. A method for identifying a molecule or compound that reduces the binding between apolipoprotein (a) and low-density lipoprotein (LDL), and inhibits the formation of lipoprotein(a) (Lp(a)), comprising:
contacting a molecule or compound of interest with a mixture of LDL and apolipoprotein (a);
determining whether the contact between the molecule or compound of interest and the mixture results in a decrease in the binding between apolipoprotein (a) and LDL, a decrease in the amount of Lp(a), or both, compared to that in a mixture without the molecule or compound of interest,
wherein a decrease in the binding between apolipoprotein (a) and LDL or a decrease in the amount of Lp(a) indicates that the molecule or compound of interest reduces the binding between apolipoprotein (a) and LDL, and inhibits the formation or reduces the amount of Lp(a).
